(19) United States
(12) Patent Application Publication
(10) Pub. No.: US 2024/0277740 A1
(43) Pub. Date: Aug. 22, 2024

(54) TREATMENT OF KNOWN AND UNKNOWN VIRAL INFECTION WITH LIPID AGENTS

(71) Applicant: Viratides, LLC, Middleton, WI (US)

(72) Inventor: Jason R. Bond, Middleton, WI (US)

(21) Appl. No.: 17/998,822

(22) PCT Filed: May 12, 2021
(86) PCT No.: PCT/US21/31960

§ 371 Date: Nov. 15, 2022

Related U.S. Application Data
(60) Provisional application No. 63/026,004, filed on May 16, 2020.
(60) Provisional application No. 63/150,651, filed on Feb. 18, 2021.
(60) Provisional application No. 63/152,075, filed on Feb. 22, 2021.
(60) Provisional application No. 63/159,844, filed on Mar. 11, 2021.
(60) Provisional application No. 63/164,074, filed on Mar. 22, 2021.
(60) Provisional application No. 63/181,730, filed on Apr. 29, 2021.
(60) Provisional application No. 63/185,431, filed on May 7, 2021.

(51) Int. Cl. A61K 31/7028; A61K 9/127; A61P 31/14; A61P 31/16; A61P 31/22

(52) U.S. Cl. CPC A61K 31/7028; A61K 9/1272; A61P 31/14; A61P 31/16; A61P 31/22

(57) ABSTRACT

The present invention provides compositions, systems, kits, and methods for treating a subject with a known or unknown enveloped or non-enveloped viral infection (e.g., an unknown virus, RSV, ADV, SARS-CoV2, CHKV, DENV, HSV-1, HSV-2, EBOV, MARV, ZIKV, or a weaponized virus) by administering or providing a composition comprising a lipid agent selected from: a sulfatide, a sulfatide analog, a ceramide, a lipid moiety comprising a ceramide, a sulfoglycolipid, a sulfogalactolipid, a glycosphingolipid, a seminolipid, or a sphingomyelin. In some embodiments, the compositions reduce lung or systemic inflammation in the subject and/or inhibit viral infection. In certain embodiments, the compositions herein are employed to stop a natural pandemic or a biological attack (e.g., with new or weaponized viruses).

The present application is a § 371 National Entry Application of PCT/US2021/031960 claims, filed May 12, 2021, which claims priority to the following U.S. Provisional applications: i) 63/026,004 filed May 16, 2020; ii) 63/150,651 filed Feb. 18, 2021; iii) 63/152,075 filed Feb. 22, 2021; iv) 63/159,844 filed Mar. 11, 2021; v) 63/164,074 filed Mar. 22, 2021; vi) 63/181,730 filed Apr. 29, 2021; and vii) 63/185,431 filed May 7, 2021; all of which are herein incorporated by reference in their entireties.

### FIELD OF THE INVENTION

The present invention provides compositions, systems, kits, and methods for treating a subject with a known or unknown enveloped or non-enveloped viral infection (e.g., an unknown virus, RSV, ADV, SARS-CoV2, CHKV, DENV, HSV-1, HSV-2, EBOV, MARV, ZIKV, or a weaponized virus) by administering or providing a composition comprising a lipid agent selected from: a sulfatide, a sulfatide analog, a ceramide, a lipid moiety comprising a ceramide, a sulfoglycolipid, a sulfogalactolipid, a glycosphingolipid, a seminolipid, or a sphingomyelin. In some embodiments, the compositions reduce lung or systemic inflammation in the subject and/or inhibit viral infection. In certain embodiments, the compositions herein are employed to stop a natural pandemic or a biological attack (e.g., with new or weaponized viruses).

### BACKGROUND

A potentially civilization ending man-made viral pandemic is likely coming in the next 10-15 years due to exponential growth of molecular biology. This is the prediction of a number of people including futurist Robert Reid, a science fiction writer and creator of the “After On” podcast. While the present number of people that are able to engineer a devastating virus is relatively small, that number is expected to grow exponentially over the next decade or two. This is because the molecular biology tools to engineer viruses, including synthetic biology, will become available to a wider and wider audience as time goes by. For example, it is possible desktop nucleic acid synthesizers capable of generating long stretches of nucleic acid could be present in people's homes at some point relatively soon. In such a case, a smart, but depressed or naive high school student, could print out a smallpox or other virus and unleash it upon the world. Such release would make the current Covid-19 pandemic look very manageable by comparison. Other scenarios involve the unintentional escape of viruses from “secure” labs, such as happened with smallpox in 1977, or natural viruses that transmit from animals to humans and kick off a worldwide pandemic. The current “broad spectrum” antivirals treat at most 2-3% of known viruses and are unlikely to stop a new or weaponized viral pandemic. As such, what is a needed is an antiviral treatment that is an inhibitor against most known viruses such that future pandemics can be stopped before it is too late.

### SUMMARY OF THE INVENTION

The present invention provides compositions, systems, kits, and methods for treating a subject with a known or unknown enveloped or non-enveloped viral infection (e.g., an unknown virus, RSV, ADV, SARS-CoV2, CHKV, DENV, HSV-1, HSV-2, EBOV, MARV, ZIKV, or a weaponized virus) by administering or providing a composition comprising a lipid agent selected from: a sulfatide, a sulfatide analog, a ceramide, a lipid moiety comprising a ceramide, a sulfoglycolipid, a sulfogalactolipid, a glycosphingolipid, a seminolipid, or a sphingomyelin. In some embodiments, the compositions reduce lung or systemic inflammation in the subject and/or inhibit viral infection. In certain embodiments, the compositions herein are employed to stop a natural pandemic or a biological attack (e.g., with new or weaponized viruses).

In some embodiments, provided herein are methods of treating a subject infected with an enveloped or non-enveloped virus comprising: administering a composition to a subject, or providing the composition to the subject such that the subject administers the composition to themselves; wherein the subject is infected with an enveloped virus (or non-enveloped virus); wherein the composition comprises a plurality of at least one type of lipid agent selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; and optionally wherein the at least one type of lipid agent is a naked lipid agent or incorporated into, or on, an artificial carrier. In certain embodiments, the administration reduces the viral load in the subject. In other embodiments, the administration reduces inflammation caused by the virus (e.g., lung, organ, or systemic inflammation). In particular embodiments, the administering or the administers: i) reduces the level (e.g., at least 10% . . . 20% . . . 30% . . . 40% . . . 80% . . . or 95%) of the enveloped or non-enveloped virus in a sample (e.g., blood, plasma, or serum sample) from the subject compared to a sample (e.g., blood, plasma, or serum) taken from the subject prior to the administering or the administers; and/or ii) reduces the level of the enveloped or non-enveloped virus in a sample from said subject such that it is undetectable or nearly undetectable. In particular embodiments, the subject has a first level of lung inflammation (or systemic inflammation), and wherein the administering or the administers reduces the level of lung inflammation (or systemic inflammation) of the subject from the first level to a second level that is lower than the first level (e.g., 10% lower . . . 20% lower . . . 30% lower . . . 40% lower . . . 50% lower . . . 60% lower . . . 70% lower . . . 80% lower . . . 90% lower . . . 95% lower . . . or removes all inflammation).

In some embodiments, provided herein are methods of treating a subject infected with an unknown virus (e.g., not differentially diagnosed) comprising: administering a composition to a subject, or providing said composition to said subject such that said subject administers said composition to themselves; wherein said subject is infected with an unknown virus causing symptoms consistent with infection from at least two viruses (e.g., at least 2, 3, 4, 5, 6, or more) from different taxonomic classifications selected from: genus, family, order, class, phylum, and kingdom; wherein said administering or said administers occurs prior to any diagnostic test to determine the species (or genus) identity of said unknown virus; wherein said composition comprises a plurality of at least one type of lipid agent selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; and wherein said at least one type of lipid agent is a naked lipid agent or incorporated into, or on, an artificial carrier. In particular embodiments the administering or the administers: i) reduces the level (e.g., at least 10% . . . 20% . . . 30% . . . 40% . . . 80% . . . or 95%) of the unknown virus in a sample (e.g., blood, plasma, or serum sample) from the subject compared to a sample (e.g., blood, plasma, or serum) taken from the subject prior to the administering or the administers; and/or ii) reduces the level of the unknown virus in a sample from said subject such that it is undetectable or nearly undetectable. In particular embodiments, the subject has a first level of lung inflammation (or systemic inflammation), and wherein the administering or the administers reduces the level of lung inflammation (or systemic inflammation) of the subject from the first level to a second level that is lower than the first level (e.g., 10% lower . . . 20% lower . . . 30% lower . . . 40% lower . . . 50% lower . . . 60% lower . . . 70% lower . . . 80% lower . . . 90% lower . . . 95% lower . . . or removes all inflammation).

In particular embodiments, provided herein are methods of treating a subject infected with a virus (e.g., unknown enveloped or non-enveloped virus) unknown to science (and otherwise known to mankind) before the year 2021 or before the year 2022 comprising: administering a composition to a subject, or providing said composition to said subject such that said subject administers said composition to themselves; wherein said subject is infected with a virus unknown to science (or mankind) before the year 2021 or the year 2022; wherein said composition comprises a plurality of at least one type of lipid agent selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; and wherein said at least one type of lipid agent is a naked lipid agent or incorporated into, or on, an artificial carrier. In particular embodiment, the administering or the administers: i) reduces the level (e.g., at least 10% . . . 20% . . . 30% . . . 40% . . . 80% . . . or 95%) of the enveloped or non-enveloped virus in a sample (e.g., blood, plasma, or serum sample) from the subject compared to a sample (e.g., blood, plasma, or serum) taken from the subject prior to the administering or the administers; and/or ii) reduces the level of the enveloped or non-enveloped virus in a sample from said subject such that it is undetectable or nearly undetectable. In particular embodiments, the subject has a first level of lung inflammation (or systemic inflammation), and wherein the administering or the administers reduces the level of lung inflammation of the subject from the first level to a second level that is lower than the first level (e.g., 10% lower . . . 20% lower . . . 30% lower . . . 40% lower . . . 50% lower . . . 60% lower . . . 70% lower . . . 80% lower . . . 90% lower . . . 95% lower . . . or removes all inflammation).

In certain embodiments, provided herein are methods comprising: shipping a container to, or receiving a container from, at least one location in a first Country's Strategic National Stockpile for viral infection response, wherein said container comprises a composition, and wherein said composition comprises a plurality of at least one type of lipid agent (e.g., in powder form) selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; wherein said at least one type of lipid agent is a naked lipid agent or incorporated into, or on, an artificial carrier. In some embodiments, the first Country's Strategic National stockpile is located in a country selected from: the United States, China, Germany, France, Great Britain, India, Canada, Japan, or Australia.

In further embodiments, provided herein are system and kits comprising: a) a composition comprising a plurality of at least one type of lipid agent selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; and b) instructions for treating said subject with said composition, wherein said subject is infected with: an unknown virus, a weaponized variant of a virus (e.g., containing non-naturally introduced changes), or a known virus.

In certain embodiments, the lipid agent comprises a sulfatide. In other embodiments, the fatty acid chain length of the sulfatide is at least 16 or at least 18 (e.g., 16:0, 16:1, 17:0, 17:1, 18:0, 18:1, 19:0, 19:1, 20:0, 20:1, 21:0, 21:1, 22:0, 22:1, 23:0, 23:1, 24:0, 24:1, etc.). In further embodiments, the fatty acid chain length of the sulfatide is at least 24 (e.g., 24, 25, 26, 27, 28, 20, or 30). In particular embodiments, the sulfatide comprises at least two different types of sulfatides (e.g., at least 2, 3, 4, 5, 6, or 7). In particular embodiments, the at least one type of sulfatide is selected from the group consisting of: 18:0(2R—OH) Sulfo GalCer; 18:0(2S—OH) Sulfo GalCer; C24:1 Mono-Sulfo Galactosyl(B) Ceramide (d18:1/24:1); C17 Mono-Sulfo Galactosyl(β) Ceramide (d18:1/17:0); C12 Mono-Sulfo Galactosyl(B) Ceramide (d18:1/12:0); C12 Di-Sulfo Galactosyl(B) Ceramide (d18:1/12:0); C24 Mono-Sulfo Galactosyl(β) Ceramide (d18:1/24:0); C24:1 Mono-sulfo galactosyl (alpha) ceramide (d18:1/24:1); C19:0-Sulfatide; N-Nonadecanoyl-sphingosyl-beta-D-galactoside-3-sulfate; and mixtures thereof. In some embodiments, the sulfatides employed with any of the embodiments described herein: i) is composed of ceramides possessing 4-sphingenine (d18:1) with C22 hydroxy FAs (C22:0 h), C23:0 h, C24:0 h, and C24:1 h and with C24 normal FAs (C24:0) and C24:1; or ii) is composed of ceramides possessing d18:1 with C16:0, C16:0 h, C18:0, C18:0 h, C20:0, C21:0, C22:1, C22:0, C21:0 h, C23:0, C26:1, and C26:0 and phytosphingosine (t18:0) with C20:0 h and C24:0 h. In certain embodiments, the lipid agent comprises the sulfatide mimic oleic acid sulfated chitosan (OlcShCs), as described in Kocabay et al., Int. J. of Bio. Macro., 147:792-798 (2020), herein incorporated by reference, including for the structure of OlcShCs).

In certain embodiments, the lipid agent comprises a sulfatide analog, wherein the sulfatide analog comprises the structure of Formula (I):

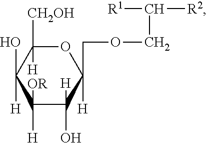

wherein R is SO₃; and wherein R¹ is —(CH₂)ₙ—CH₃ where n is an integer from 10 to 40 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40). In some embodiments, the n is an integer from 20-30.

In certain embodiments, the lipid agent comprises a sulfatide analog, wherein the sulfatide analog comprises the structure of Formula (II):

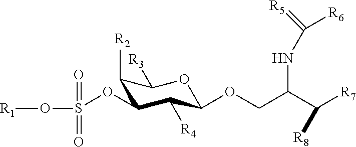

wherein R₁ is selected from the group consisting of a bond, a hydrogen, a C₁ to C₃₀ alkyl, C₁ to C₃₀ substituted alkyl, a C₁ to C₃₀ alkenyl, a C₁ to C₃₀ substituted alkenyl and a C₅ to C₁₂ sugar; R₂ is selected from the group consisting of a hydrogen, a hydroxy group, a methoxy group, and an alkoxy group; R₃ is selected from the group consisting of a hydrogen, a hydroxy group, a methoxy group, an ethoxy group, and an alkoxy group; R₄ is selected from the group consisting of a hydrogen, a hydroxy group and an alkoxy group; R₅ is selected from the group consisting of a hydrogen, a hydroxyl, a carbonyl, an alkoxy and a bond; R₆ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; R₇ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₈ is selected from the group consisting of a hydrogen, a hydroxyl group, a carbonyl, an alkoxy group and a bond.

In some embodiments, the lipid agent comprises a sulfatide analog, wherein the sulfatide analog comprises the structure of Formula (III):

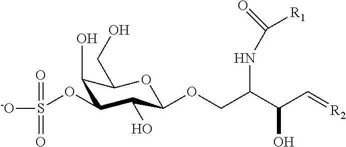

wherein R₁ is selected from the group consisting of a C₁ to C₄₀ alkyl, a C₁ to C₄₀ substituted alkyl, a C₁ to C₄₀ alkenyl, a C₁ to C₄₀ substituted alkenyl and a C₁ to C₄₀ alkynl; and R₂ is selected from the group consisting of a hydrogen, a hydroxyl group, a carbonyl, an alkoxy group and a bond.

In further embodiments, the lipid agent comprises a sulfatide analog, wherein the sulfatide analog comprises the following structure:

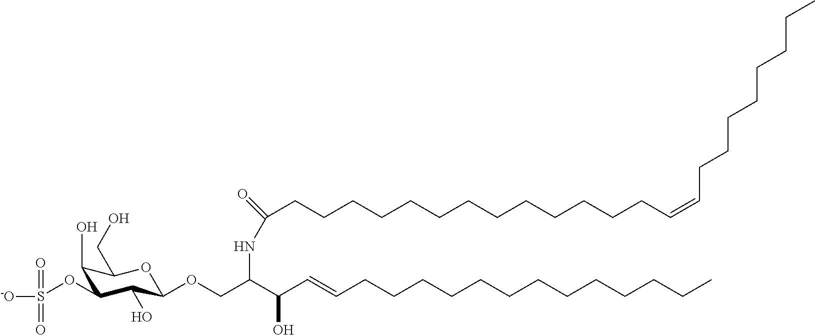

In some embodiments, the at least one lipid agent comprises said sulfatide. In particular embodiments, the fatty acid chain length of said sulfatide is selected from the group consisting of: 16, 17, 18, 19, 20, 21, 22, 23, or 24, and optionally wherein said composition contains only one type of sulfatide, or only two types of sulfatides, and is detectably free of other types of sulfatides. In other embodiments, the subject has lung inflammation or vascular inflammation, and wherein said administering or administers reduces said lung inflammation and/or said vascular inflammation. In other embodiments, the at least one lipid agent is a sulfatide, wherein said sulfatide is one that does not serve as an auto-antigen for multiple sclerosis when administered to a human, and/or wherein said sulfatide causes inflammation reduction in said subject and/or does not cause coagulation, and/or does not cause cancer metastasis when administered to a subject. In further embodiments, the at least one lipid agent is sulfatide but is not C24:1, C26:1, or C26:1, and said composition is detectably free of C24:1, C26:1, or C26:1. In certain embodiments, the at least one lipid agent is a sulfatide, and wherein said composition comprises only one, only two, types of sulfatides, and is detectably free of any other type of sulfatide.

In particular embodiments, the lipid agent comprises a glycosphingolipid. In other embodiments, the glycosphingolipid comprises a ganglioside. In additional embodiments, the glycosphingolipid comprises a glucosylceramide. In further embodiments, the glycosphingolipid comprises a galactosylceramide.

In some embodiments, provided herein are articles of manufacture comprising an orally ingestible pill or capsule, wherein the orally ingestible pill or capsule comprises: a) a composition comprising a plurality of at least one type of lipid agent (or only one type of lipid agent and not others) selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; wherein said at least one type of lipid agent is a naked lipid agent or incorporated into, or on, an artificial carrier; and b) an enteric coating which surrounds said composition. In certain embodiments, the pill or capsule comprises a capsule, wherein said capsule comprises a softgel. In other embodiments, the softgel comprises gelatin. In further embodiments, the composition further comprises a solvent (e.g., DMSO).

In some embodiments, the virus is SARS-CoV-2 or SARS-CoV-1. In certain embodiments, the virus is a non-enveloped virus (e.g., Norovirus, Rhinovirus, or Poliovirus). In further embodiments, the enveloped or non-enveloped virus is a respiratory virus. In particular embodiments, the virus is SARS-CoV2, HSV-1, HSV-2, HBV, HCV, or RSV. In certain embodiments, the enveloped or non-enveloped virus is HBV, HCV, RSV, HSV-1, HSV-2, ADV, Ebola, Marburg virus, Dengue virus serotype 1, Dengue virus serotype 2, Dengue virus serotype 3, Dengue virus serotype 4, Zika virus, Chikungunya (CHIKV), human cytomegalovirus (HCMV), human adenovirus (ADV), herpes zoster (shingles), or a weaponized variant of any of said viruses. In other embodiments, the virus is a coronavirus. In additional embodiments, the virus is influenza A virus. In further embodiments, the virus is human immunodeficiency virus type 1 (HIV-1) or HIV-2. In certain embodiments, the virus is selected from the group consisting of: Lassa fever virus, lymphocytic choriomeningitis virus, Ebola virus, Marburg virus, hepatitis B virus, Herpes simplex virus type 1, Herpes simplex virus type 2, cytomegalovirus, Simian virus, type 5, Mumps virus, avian sarcoma leucosis virus, human T-lymphotropic virus, type 1, equine infectious anemia virus, Sandfly fever Naples phlebovirus (SFNV), classical swine fever virus (CSFV), Infectious hematopoietic necrosis virus (IHNV), Porcine reproductive and respiratory syndrome (PRRS), viral hemorrhagic septicemia virus (VHSV), Newcastle disease virus (NDV), Porcine epidemic diarrhea virus (PEDV), vesicular stomatitis virus, and rabies virus. In certain embodiments, the virus is selected from the group consisting of: bovine viral diarrhea virus (BVDV), measles virus, Human metapneumovirus, rhinovirus, and yellow fever virus. In particular embodiments, the virus is selected from the group consisting of: tomato spotted wilt virus, Tomato yellow leaf curl virus (TYLCuV), or a member of Emaravirus, Bunyavirus, and Rhabdovirus. In certain embodiments, the virus is selected from: Actinidia chlorotic ringspot-associated emaravirus, Blackberry leaf mottle associated emaravirus, European mountain ash ringspot-associated emaravirus, Fig mosaic emaravirus, High Plains wheat mosaic emaravirus, Pigeonpea sterility mosaic emaravirus 1, Pigeonpea sterility mosaic emaravirus 2, Pistacia emaravirus B, Raspberry leaf blotch emaravirus, Redbud yellow ringspot-associated emaravirus, variola virus, Hantavirus, Rose rosette emaravirus, California encephalitis virus, La Crosse encephalitis virus, Jamestown Canyon virus, and Snowshoe hare virus vector.

In some embodiments, the lipid agent is a naked lipid agent. In other embodiments, the lipid agent is incorporated into, or on, an artificial carrier. In certain embodiments, the artificial carrier comprises a liposome, nanoparticle (e.g., PLGA), dendrimer, quantum dot, polymersome, gold nanoparticle, or carbon nanotube. In other embodiments, the artificial carrier comprises a multilamellar vesicle (MLV), a small unilamellar liposome vesicle (SUV), micelle, and/or large unilamellar vesicles (LUV). In certain embodiments, the subject is a human or animal, such as a dog, cat, horse, cow, pig, or other livestock.

In particular embodiments, the administering (e.g., for intravenous administration) is such that the subject receives about 0.1-15 mg or 0.5-4.0 or 0.4-10 mg of the lipid agent per kilogram of the subject (e.g., 0.1 . . . 0.5 . . . 1.0 . . . 1.5 . . . 2.0 . . . 2.5 . . . 3.0 . . . 3.5 . . . 4.0 . . . 5.0 . . . 6.0 . . . 7.0 . . . 8.0 . . . 9.0 . . . 10 . . . or 15 mg per kg) 1-5 times per day (e.g., for 4-20 days, such as 6-7 days). In further embodiments, the administering is such that the subject receives about 0.1-20 or 0.01-50 mg of the lipid agent per kilogram of the patient (e.g., 0.01 . . . 0.05 . . . 0.1 . . . 0.5 . . . 1.0 . . . 5.0 . . . 8.0 . . . 10.0 . . . 12.0 . . . 15.0 . . . 18.0 . . . 20.0 . . . 25 . . . 30 . . . 35 . . . 40 . . . or 50 mg per kg) 1-5 times per day (e.g., for 2-30 days, such as 5, 6, 7, 8, 9, 10 . . . 20 . . . 25 . . . or 30 days).

In further embodiments (e.g., for oral administration) the administering (e.g., patient swallowing a pill or capsule containing the lipid agent (e.g., sulfatide), or taking liquid beverage infused with the lipid agent (e.g., sulfatide)) is such that the subject receiving between about 5 mg to 1500 mg per kilogram of patient (e.g., 5 . . . 150 . . . 300 . . . 500 . . . 750 . . . 1000 . . . 1250 . . . or 1500), 1-5 times per days. In some embodiments, the dosage form is a pill (e.g., lipid agent powder, such as sulfatide powder with or without binders or fillers, pressed into the form of a pill, which may have an enteric coating), or is the form of a capsule (e.g., containing a liquid with sulfatide present, which may have an enteric coating). In some embodiments, the subject is administered, or administers to themselves (e.g., pill, capsule, or inhaler) sulfatides or other lipid agent every day for long term treatment (e.g., everyday for 21 days or everyday for a month, or everyday for 6 months).

In some embodiments, the administering is intravenous administration. In other embodiments, the administration is topical (e.g., in a cream or salve, such as for treating HSV-1). In further embodiments, the administering is via the subject's airway. In certain embodiments, the administration is oral (e.g., in a gel cap, pill, or similar dosage form, which may have an enteric coating). In some embodiments, the oral administration is 10-1000 mg of sulfatides per kilogram of subject (e.g., 10 . . . 75 . . . 100 . . . 125 . . . 150 . . . 200 . . . 250 . . . 300 . . . 400 . . . 500 . . . 650 . . . 800 . . . 1000 mg/kg). In additional embodiments, the composition is freeze-dried, or in micro-droplets, or a powder, and administering or providing is via the subject's airway, or provided in a nebulizer or other airway administration device. In other embodiments, administering or providing an anti-coagulant to the subject. In certain embodiments, the methods further comprise: administering or providing a different anti-viral agent to the subject.

In some embodiments, the subject has lung inflammation, organ inflammation, vascular, or systemic inflammation. In certain embodiments, the subject is on a ventilator. In other embodiments, the subject has general body inflammation. In other embodiments, the composition further comprises a physiologically tolerable buffer or IV solution. In certain embodiments, the administering or administers reduces the lung inflammation (e.g., in a patient infected with a respiratory virus, such as RSV or ADV).

In particular embodiments, provided herein are systems, kits, and articles of manufacture comprising: a) a composition comprises a plurality of at least one type of lipid agent selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; wherein, optionally, the at least one type of lipid agent is a naked lipid agent or incorporated into, or on, an artificial carrier; and b) a medical container selected from the group consisting of: i) an IV fluid solution bag, ii) a syringe vial, iii) a syringe, iv) a sterile shipping container configured for shipping powder or liquid, v) an airway administration device, and vi) an orally ingestible dosage form (e.g., pill or capsule, which may have an enteric coating).

In some embodiments, the systems, kits, and articles of manufacture, further comprise a physiologically tolerable buffer or intravenous fluid. In further embodiments, the composition is present inside the medical container. In additional embodiments, the composition is a liquid. In further embodiments, the composition is in a powder form.

In some embodiments, the medical container is the airway administration device, wherein the airway administration device is a nebulizer. In further embodiments, the medical container is the IV solution bag, wherein the composition is present in the IV solution bag, and wherein the composition further comprises an IV fluid. In additional embodiments, the medical container is the syringe vial, wherein the composition is present in the syringe vial, and wherein the composition further comprises a physiological tolerable buffer. In other embodiments, the medical container is the sterile shipping container, wherein the composition is present in the sterile shipping container. In further embodiments, the composition is in the form of a powder. In particular embodiments, the composition is in the form of a liquid.

In certain embodiments, provided herein are in vitro compositions comprising: a) a plurality of at least one type of lipid agent (or only one and not others) selected from the group consisting of: a sulfatide, a sulfatide analog, a ceramide, a lipid moiety comprising a ceramide, a sulfoglycolipid, a sulfogalactolipid, a glycosphingolipid, a seminolipid, and a sphingomyelin; wherein, optionally, the at least one type of lipid agent is a naked lipid agent or incorporated into, or on, an artificial carrier; and b) an enveloped or non-enveloped virus. In some embodiments, the enveloped or non-enveloped virus is selected from: SARS-CoV-2, a respiratory virus, a coronavirus, influenza virus, adenovirus, human immunodeficiency virus type 1 (HIV-1), Lassa fever virus, lymphocytic choriomeningitis virus, Ebola virus, Marburg virus, hepatitis B virus, Herpes simplex virus type 1, Herpes simplex virus type 2, cytomegalovirus, Simian virus, type 5, Mumps virus, avian sarcoma leucosis virus, human T-lymphotropic virus, type 1, coxsackieviruses, rotavirus, or poliovirus, equine infectious anemia virus, vesicular stomatitis virus, and rabies virus.

In some embodiments, provided herein are methods of treating a subject with a viral infection comprising: administering a composition to a subject, or providing the composition to the subject such that the subject administers the composition to themselves, wherein the subject is infected with a virus, and wherein the composition comprises a plurality of at least one type of sulfatide or at least one type of sulfatide analog.

In certain embodiments, the virus is SARS-CoV-2. In some embodiments, wherein said virus is an enveloped virus or a non-enveloped virus. In other embodiments, the virus is a respiratory virus (e.g., MERS, SARS-CoV-1, SARS-CoV-2, adenovirus, enterovirus, rhinovirus, Human metapneumovirus, Influenza Virus, Parainfluenza virus, and Respiratory Syncytial Virus (RSV)). In certain embodiments, the virus is a coronavirus.

In some embodiments, the compositions further comprise non-sulfatide lipids, wherein the non-sulfatide lipids and the plurality of at least one type of sulfatide are combined in the form of a plurality of sulfatide-containing liposomes. In certain embodiments, the non-sulfatide lipids comprise phospholipids. In other embodiments, the non-sulfatide lipids comprises phosphatidylcholine. In other embodiments, the non-sulfatide lipids are selected from one or more of the group consisting of: distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); and 1,2-Dipalmitoyl-sn-glycero-3-phosphate (DPPA). In additional embodiments, the at least one type of sulfatide not incorporated into part of a larger molecular structure (naked sulfatide).

In particular embodiments, each of the plurality of sulfatide-containing liposomes comprise about 10-40% of the at least one type of sulfatide and about 60-90% of the lipids (e.g., about 10:90% . . . 20:80% . . . 30:70% . . . 40:60%). In other embodiments, each of the plurality of sulfatide-containing liposomes comprise about 40-65% of the at least one type of sulfatide and about 35-60% of the lipids (e.g., about 40:60% . . . 50:50% . . . 60:40% . . . 65:35%). In further embodiments, each of the plurality of sulfatide-containing liposomes comprises about 0.5-40% cholesterol or cholesterol sulfate (e.g., about 0.5% . . . 4.0% . . . 10% . . . 20% . . . 30% . . . or 40%).

In certain embodiments, the at least one type of sulfatide comprises a fatty acid with a chain length of 12-24 carbon atoms (e.g., 12, 13, 14, 15, 16, 17, 18, 19, 20, 21, 22, 23, or 24). In particular embodiments, the chain length is 18. In some embodiments, the chain length 12, 17, or 24. In additional embodiments, the at least one type of sulfatide or sulfatide analog is at least two different types of sulfatides or sulfatide analogs (e.g., 2, 3, 4, 5, 6, or more). In particular embodiments, the at least one type of sulfatide is selected from the group consisting of: 18:0(2R—OH) Sulfo GalCer; 18:0(2S—OH) Sulfo GalCer; C24:1 Mono-Sulfo Galactosyl(β) Ceramide (d18:1/24:1); C17 Mono-Sulfo Galactosyl(β) Ceramide (d18:1/17:0); C12 Mono-Sulfo Galactosyl(B) Ceramide (d18:1/12:0); C12 Di-Sulfo Galactosyl(B) Ceramide (d18:1/12:0); C24 Mono-Sulfo Galactosyl(B) Ceramide (d18:1/24:0); C24:1 Mono-sulfo galactosyl (alpha) ceramide (d18:1/24:1); C19:0-Sulfatide; N-Nonadecanoyl-sphingosyl-beta-D-galactoside-3-sulfate; and mixtures thereof.

In some embodiments, the sulfatide-containing liposomes comprise multilamellar vesicles (MLVs), small unilamellar liposome vesicles (SUVs), and/or large unilamellar vesicles (LUVs). In certain embodiments, the subject is a human or an animal (e.g., livestock).

In further embodiments, the administering is such that the subject receives about 0.5-4.0 mg or 0.1-20 mg of the sulfatide or sulfatide analog per kilogram of the patient (e.g., 0.5 . . . 1.0 . . . 1.5 . . . 2.0 . . . 2.5 . . . 3.0 . . . 3.5 . . . 4.0 mg per kg) 1-5 times per day. In particular embodiments, the sulfatide analog comprises the structure of Formula (I):

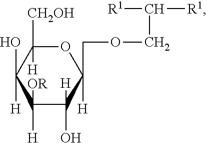

wherein R is SO₃; and
wherein R¹ is —(CH₂)ₙ—CH₃ where n is an integer from 10 to 30 or 10 to 40 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40). In certain embodiments, the n is an integer from 20-30.

In particular embodiments, the administering is intravenous administration. In other embodiments, the administering is via the subject's airway. In additional embodiments, the composition is freeze-dried and administered via the subject's airway, or provided in a nebulizer. In other embodiments, the methods further comprise: administering or providing an anti-coagulant to the subject. In some embodiments, the methods further comprise: administering or providing an anti-viral agent to the subject.

In particular embodiments, the subject has lung inflammation. In other embodiments, the subject is on a ventilator. In some embodiments, the subject has general body inflammation. In certain embodiments, the composition further comprises a physiologically tolerable buffer (e.g., IV fluid lactated Ringer's or Hartmann's solution).

In some embodiments, provided herein are compositions comprising: a) a physiologically tolerable buffer (e.g., IV fluid lactated Ringer's or Hartmann's solution), and b) a plurality of sulfatide-containing liposomes, wherein the plurality of sulfatide-containing liposomes comprises: i) lipids, and ii) a plurality of at least one type of sulfatide or sulfatide analog, and wherein each of the plurality of sulfatide-containing liposomes comprise about 40-65% of the at least one type of sulfatide and about 35-60% of the lipids (e.g., about 40:60% . . . 50:50% . . . 60:40% . . . 65:35%).

In additional embodiments, each of the plurality of sulfatide-containing liposomes comprises 40-50% of the at least one type of sulfatide or sulfatide analog (e.g., 41 . . . 44 . . . 47 . . . 50%). In certain embodiments, the lipids comprise phospholipids. In some embodiments, the lipids comprises phosphatidylcholine. In additional embodiments, the lipids are selected from the group consisting of: distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); and 1,2-Dipalmitoyl-sn-glycero-3-phosphate (DPPA). In certain embodiments, each of the plurality of sulfatide-containing liposomes comprises about 0.5-40% cholesterol or cholesterol sulfate (e.g., 0.5 . . . 10 . . . 20 . . . 30 . . . or 40%).

In some embodiments, the at least one type of sulfatide comprises a fatty acid with a chain length of 12-24 carbon atoms (e.g., 12, 13, 14, 15, 16, 17, 18, 19, 20, 21, 22, 23, or 24). In other embodiments, the chain length is 18. In additional embodiments, the chain length 12, 17, or 24. In further embodiments, the at least one type of sulfatide is at least two different types of sulfatides (e.g., 2, 3, 4, 5, 6, or more). In particular embodiments, the at least one type of sulfatide is selected from the group consisting of: 18:0(2R—OH) Sulfo GalCer; 18:0(2S—OH) Sulfo GalCer; C24:1 Mono-Sulfo Galactosyl(B) Ceramide (d18:1/24:1); C17 Mono-Sulfo Galactosyl(β) Ceramide (d18:1/17:0); C12 Mono-Sulfo Galactosyl(B) Ceramide (d18:1/12:0); C12 Di-Sulfo Galactosyl(β) Ceramide (d18:1/12:0); C24 Mono-Sulfo Galactosyl(B) Ceramide (d18:1/24:0); C24:1 Mono-sulfo galactosyl (alpha) ceramide (d18:1/24:1); C19:0-Sulfatide; N-Nonadecanoyl-sphingosyl-beta-D-galactoside-3-sulfate; and mixtures thereof.

In further embodiments, the sulfatide-containing liposomes comprise multilamellar vesicles (MLVs), small unilamellar liposome vesicles (SUVs), and/or large unilamellar vesicles (LUVs). In other embodiments, between 40 mg and 600 mg, or 10 mg to 1200 mg (e.g., 10 . . . 150 . . . 400 . . . 600 . . . 900 . . . 1200 mg) of the sulfatide or sulfatide analog are present in the composition. In certain embodiments, the sulfatide analog comprises the structure of Formula (I):

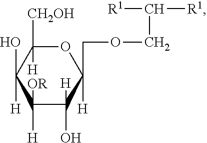

wherein R is SO₃; and wherein R¹ is —(CH₂)ₙ—CH₃ where n is an integer from 10 to 30 or 10 to 40 (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40). In further embodiments, n is an integer from 20-30.

In certain embodiments, the sulfatide-containing liposomes are freeze-dried. In other embodiments, the compositions further comprise an anti-coagulant and/or anti-viral agent.

In certain embodiments, provided herein are methods of treating a subject with a viral infection comprising: administering the composition described herein, or providing the composition described herein, to the subject such that the subject administers the composition to themselves, wherein the subject is infected with a virus.

### DESCRIPTION OF THE FIGURES

The patent or application file contains at least one drawing executed in color. Copies of this patent or patent application publication with color drawings will be provided by the Office upon request and payment of the necessary fee.

FIG. 1A shows the general structure for certain exemplary sulfatides. Sulfatides (also known as 3-O-sulfogalactosylceramide, SM4, or sulfated galactocerebroside) are sulfolipids, specifically a class of sulfoglycolipids. Sulfatides are composed of a sulphated galactose group attached to ceramide. The ceramide moiety in FIG. 1 is composed of a sphingosine base (dihydroxy sphingosine, d18:1) and a fatty acid with a chain length of 16-24 carbon atoms (the fatty acid could also have a chain length of 12-15 or 16-24 in certain embodiments). The asterisk in FIG. 1 represents a possible position of hydroxylation. Sulfatides occur in the extracellular leaflet of the plasma membrane of many cells in eukaryotic organisms. Sulfatide is one of the major lipids expressed in the central nervous system and is also present in the lung, trachea, kidney, spleen, platelets, testis, circulating blood, and the gastrointestinal tract. FIG. 1B shows the structure of sulfatide, d18:1-C24:0 h (note, C:24 could instead be C: 16, C: 17, C: 18, C: 19, C:20, C:21, C:22, or C:23). FIG. 1C shows the structure of t18:0-C24:0 h (note, C:24 could instead be C:16, C:17, C:18, C:19, C:20, C:21, C:22, or C:23).

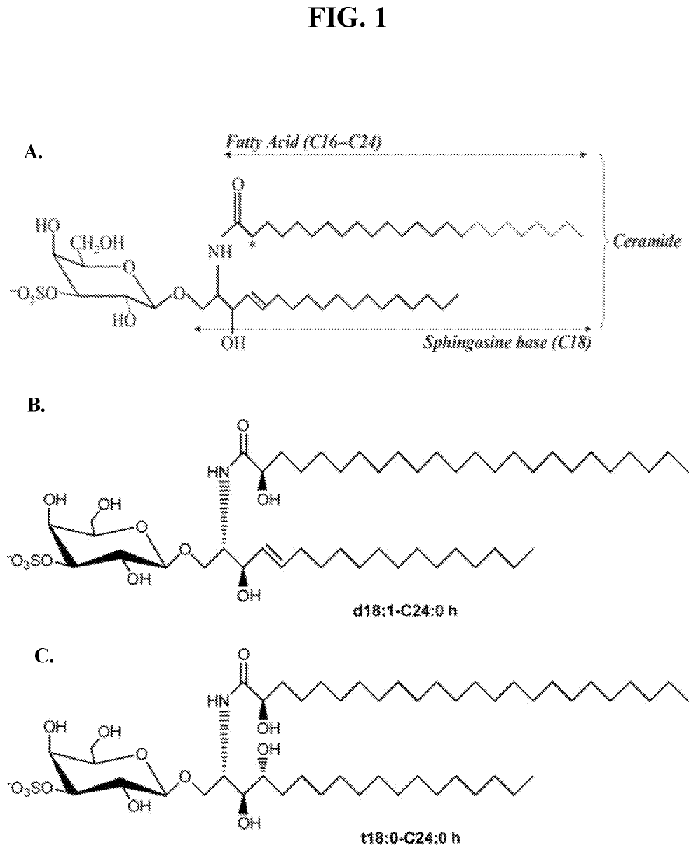

FIG. 2 shows the chemical structure of three exemplary gangliosides: GM1, GM2, and GM3.

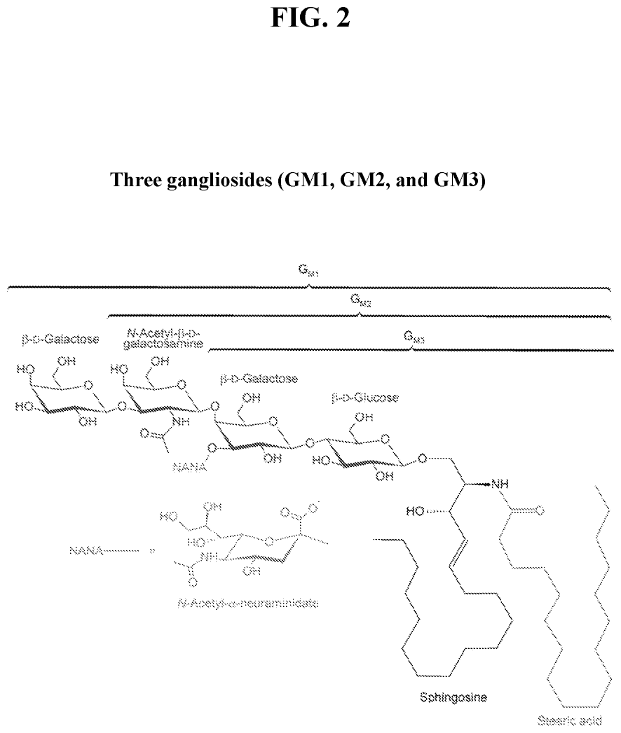

FIG. 3 shows the chemical structure of nine exemplary ceramides: ceramide (EOS) (ceramide 1); ceramide (NS) (ceramide 2); ceramide (NP) (ceramide 3); ceramide (EOH) (ceramide 4); ceramide (AS) (ceramide 5); ceramide (NH) (ceramide 6); ceramide (AP) (ceramide 7); ceramide (AH) (ceramide 8); and ceramide (EOP) (ceramide 9).

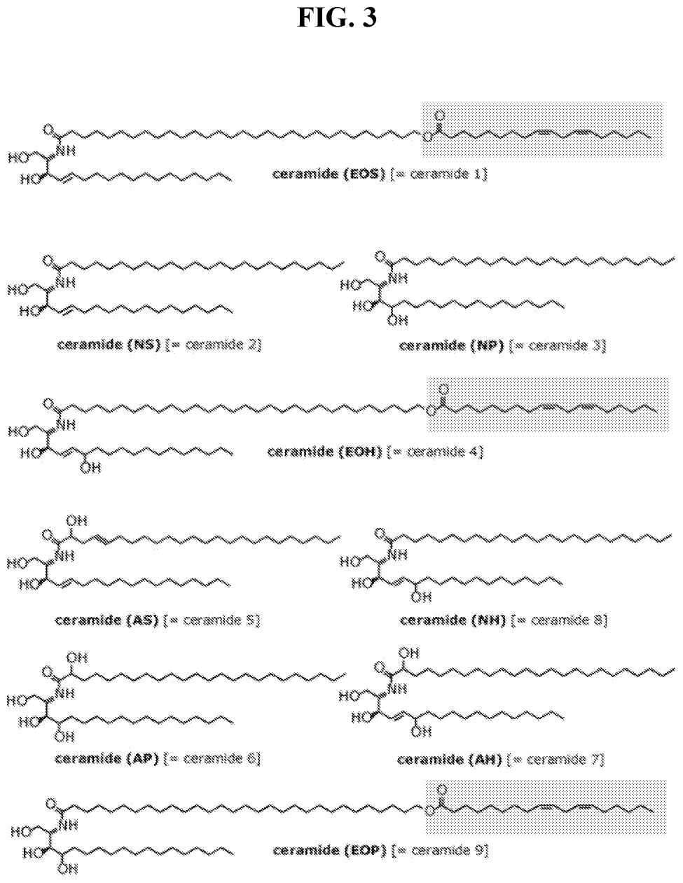

FIG. 4 shows the basic chemical structure of glycosphingolipids.

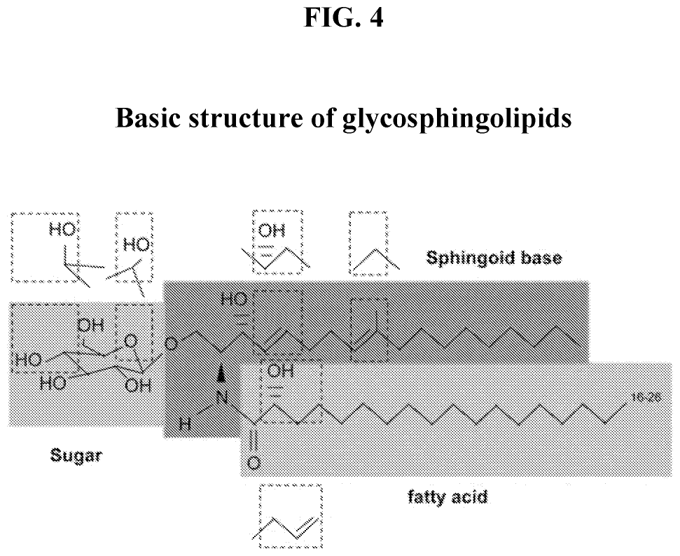

FIG. 5 shows the chemical structure of galactosylceramides, where the fatty acid chain can vary in length compared to what is shown in this figure.

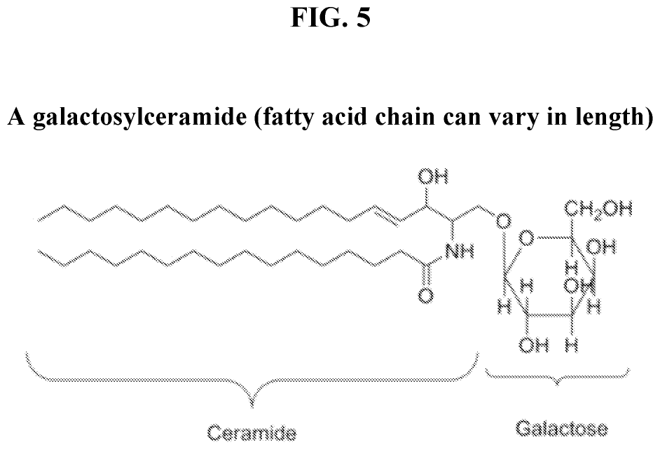

FIG. 6A shows the chemical structure of a glucosylceramide, where the fatty acid chain length can vary compared to what is shown in this figure. FIG. 6b shows glucosylceramides 1-5.

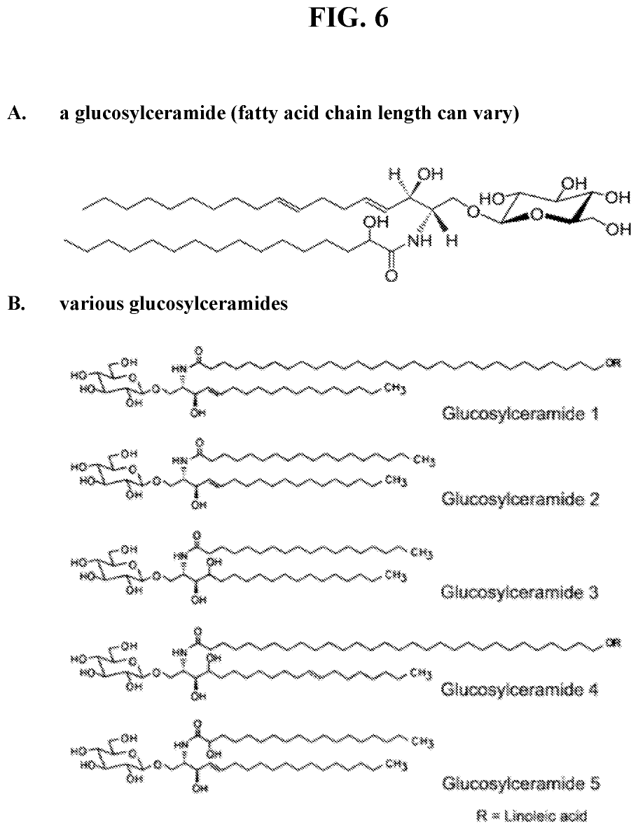

FIG. 7A shows a first exemplary sphingomyelin, and FIG. 7B shows a second exemplary sphingomyelin.

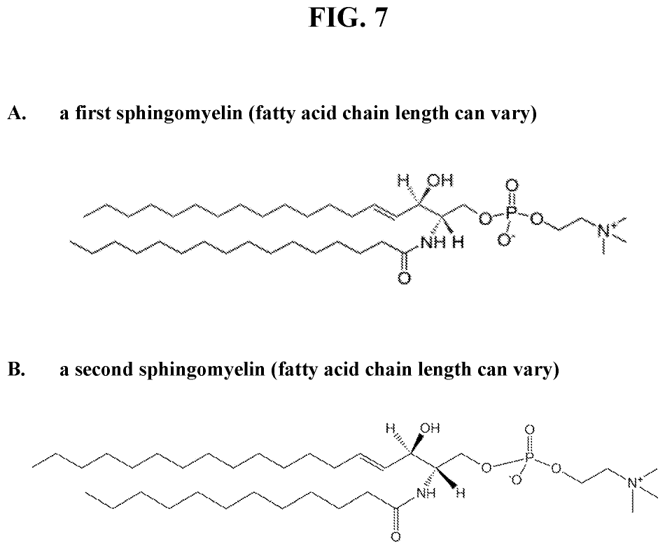

FIG. 8 shows the chemical structure of sulfatide C24:1 3′-sulfo Galactosylceramide (d18:1/24:1(15Z)).

FIG. 9A shows cytotoxicity results (blue line) and antiviral inhibition results (green line) for ADV-5 (Adenovirus), and FIG. 9B shows cytotoxicity results (blue line) and antiviral inhibition results (green line) for CHKV (Chikungunya virus).

FIG. 10A shows cytotoxicity results (blue line) and antiviral inhibition results (green line) for DENV (Dengue virus), and FIG. 10B shows cytotoxicity results (blue line) and antiviral inhibition results (green line) for HSV-1 (Herpes simplex virus, type 1).

FIG. 11A shows cytotoxicity results (blue line) and antiviral inhibition results (green line) for HSV-2 (Herpes simplex virus, type 2), and FIG. 11B shows cytotoxicity results (blue line) and antiviral inhibition results (green line) for INFV B (Influenza A).

FIG. 12A shows cytotoxicity results (blue line) and antiviral inhibition results (green line) for RSV (Respiratory syncytial virus), and FIG. 12B shows cytotoxicity results (blue line) and antiviral inhibition results (green line) for pseudo EBOV (Pseudovirus VSV-EBOLA virus).

FIG. 13A shows cytotoxicity results (blue line) and antiviral inhibition results (green line) for pseudo MARV (VSV-Marburg virus), and FIG. 13B shows cytotoxicity results (blue line) and antiviral inhibition results (green line) for VEEV (Venezuelan equine encephalitis virus).

FIG. 14A shows cytotoxicity results (blue line) and antiviral inhibition results (green line) for pseudo ZIKV (Zika virus). FIG. 14B shows cytotoxicity results (blue line) and antiviral inhibition results (green line) for hCMV (human cytomegalovirus).

FIG. 15 shows the results of inhibition by the sulfatide of SARS-CoV-2-induced CPE (A540). Cell viability was monitored to determine the virus induced-CPE. Data is shown as raw A540 values in wells containing Vero E6 cells infected in the presence of either vehicle alone or varying concentrations of test-item (average of duplicates with standard deviation). Uninfected cells are shown as “Mock.” Background levels are shown in wells without cells (“no cells”). Also included, the dose-response observed with GS-441524 (single data-points).

FIG. 16 shows inhibition by the test item (sulfatide) of the CPE mediated by SARS-CoV-2 (percentage values). Values show the inhibition of the SARS-CoV-2 induced CPE, as a surrogate marker for virus replication. Data was analyzed as shown in Table 7, with values normalized to the A540 values observed in uninfected cells after subtraction of the average absorbance observed in infected cells in the presence of vehicle. Values in uninfected cells (“mock”) are included for comparison (100% inhibition). Data plotted for test-item shows the average and standard deviation of duplicates. Also included, the dose-response observed with GS-441524 (single data-points).

FIG. 17 shows IC50 values for inhibition of SARS-CoV-2 CPE by sulfatide (FIG. 17A) and GS-441524 (FIG. 17B). Values indicate the percentage inhibition of the CPE induced by live SARS-CoV-2 (MEX-BC2/2020), as compared to samples incubated with no test-item (vehicle alone). Test-item results show the average of duplicate data points. Bottom graphs show single data points for GS-441524. Data was modeled to a sigmoidal function using GraphPad Prism software fitting a dose-response curve with a variable slope (four parameters). IC50 value for GS-441524 curve was estimated by the GraphPad Prism Software with an R2=0.977. IC50 values are also summarized in Table 5.

FIG. 18 shows viability in uninfected Vero E6 cells (percentage values). Results show the extent of cell viability as determined by the neutral red uptake assay (A540) after 4 days. Data is normalized to the values observed in cells in the absence of sulfatide (“vehicle,” medium only). Test-item results show the average of duplicate data points with the standard deviation (s.d.). Average and standard deviation values for cells treated with vehicle (medium only) are derived from six replicates.

FIG. 19 shows CC50 values for Vero E6 cell viability in the presence of sulfatide (percentage values). Values indicate the percent viability estimated as percentage of that observed in samples incubated with vehicle alone (medium). Results show the average of duplicate data points. Bottom graph shows overlapping curves from sulfatide. Data was adjusted to a sigmoid function when possible, and CC50 values were calculated using GraphPad Prism software fitting a dose-response curve with a variable slope (four parameters). CC50 values are also summarized in Table 5.

FIG. 20 shows Compounds 2-6 from Kretzschmar et al., Tetrahedron, 54 (50), 10 Dec. 1998, 15189-15198 (herein incorporated by reference), which could be used as lipid agents in the methods, systems, and compositions herein. Compounds 2 is sulfonoquinovosyl dipalmitoyl glyceride (SQDG), and Compounds 3-5 are sulfatide and SQDG-mimetics. In certain embodiments, one or both fatty acid chains in Compounds 2-6 are each, independently, made shorter or longer (e.g., shorter by 5 carbons or longer by 5 carbons).

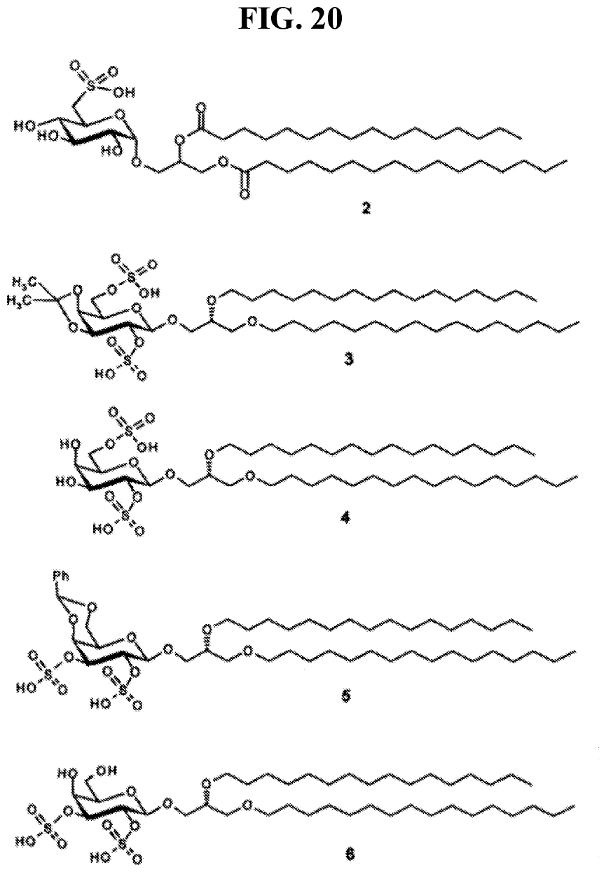

### DEFINITIONS

As used herein, the term “naked” in regard to lipid agent” refers to the lipid agents as described herein (e.g., a sulfatide, a sulfatide analog, a ceramide, a lipid moiety comprising a ceramide, a sulfoglycolipid, a sulfogalactolipid, a glycosphingolipid, a seminolipid, or a sphingomyelin) where such lipid agents are not associated with macromolecular molecules (e.g., proteins) or cell structures (e.g., cell rafts, cell membranes, etc.) that they may be, for example, associated with in nature.

### DETAILED DESCRIPTION

The present invention provides compositions, systems, kits, and methods for treating a subject with a known or unknown enveloped or non-enveloped viral infection (e.g., an unknown virus, RSV, ADV, SARS-CoV2, CHKV, DENV, HSV-1, HSV-2, EBOV, MARV, ZIKV, or a weaponized virus) by administering or providing a composition comprising a lipid agent selected from: a sulfatide, a sulfatide analog, a ceramide, a lipid moiety comprising a ceramide, a sulfoglycolipid, a sulfogalactolipid, a glycosphingolipid, a seminolipid, or a sphingomyelin. In some embodiments, the compositions reduce lung or systemic inflammation in the subject and/or inhibit viral infection. In certain embodiments, the compositions herein are employed to stop a natural pandemic or a biological attack (e.g., with new or weaponized viruses).

Examples of enveloped and non-enveloped viruses treated with the methods provided herein include, but are not limited to, the following viral families and species: Retroviridae (e.g., HIV (such as HIV1 and HIV2), MLV, SIV, FIV, Human T-cell leukemia viruses 1 and 2, XMRV, and Coltiviruses (such as CTFV or Banna virus)); Togaviridae (for example, alphaviruses (such as Ross River virus, Sindbis virus, Semliki Forest Virus, O'nyong'nyong virus, Chikungunya virus, Eastern equine encephalitis virus, Western equine encephalitis virus) or rubella viruses); Flaviridae (for example, dengue viruses, encephalitis viruses (such as West Nile virus or Japanese encephalitis virus), yellow fever viruses); Coronaviridae (for example, coronaviruses such as SARS virus, SARS-Cov-2, or Toroviruses); Rhabdoviridae (for example, vesicular stomatitis viruses, rabies viruses); Paramyxoviridae (for example, parainfluenza viruses, mumps virus, measles virus, respiratory syncytial virus, sendai virus, and metopneumovirus); Orthomyxoviridae (for example, influenza viruses); Bunyaviridae (for example, Hantaan virus, bunya viruses (such as La Crosse virus), phleboviruses, and Nairo viruses); Hepadnaviridae (Hepatitis B viruses); Herpesviridae (herpes simplex virus (HSV-1 and HSV-2), varicella zoster virus, cytomegalovirus (CMV), HHV-8, HHV-6, HHV-7, and pseudorabies virus); Filoviridae (filoviruses including Ebola virus and Marburg virus) and Poxviridae (variola viruses, vaccinia viruses, pox viruses (such as smallpox, monkey pox, and Molluscum contagiosum virus), yatabox virus (such as Tanapox and Yabapox)). In certain embodiments, the enveloped and viruses include herpes virus, influenza virus, paramyxovirus, respiratory syncytial virus, corona virus, HIV, hepatitis B virus, hepatitis C virus, SARS-CoV virus, and SARS-CoV-2 virus. In some embodiments, the enveloped and non-enveloped virus is selected from the group consisting of: Lassa fever virus, lymphocytic choriomeningitis virus, Ebola virus, avian IAV (H5N1), Adenovirus, Marburg virus, hepatitis B virus, Herpes simplex virus, type 1, Herpes simplex virus, type 2, cytomegalovirus, Simian virus, type 5, Mumps virus, avian sarcoma leucosis virus, human immunodeficiency virus, type 1, human T-lymphotropic virus, type 1, equine infectious anemia virus, vesicular stomatitis virus, rabies virus, and combinations thereof. In further embodiments, the virus is selected from the group consisting of: Sindbis virus, Rubella virus, Yellow fever virus, Hepatitis C virus, Influenza virus, Measles virus, Mumps virus, Human Metapneumovirus, Respiratory Syncytial virus, Vesicular Stomatitis virus, Rabies virus, Hantaan virus, Crimean-Congo Hemorrhagic fever virus, Rift Valley fever virus, Coronavirus, SARS virus, LCM virus, human T-cell leukemia virus, human immune deficiency virus (HIV), marburg virus, Ebola virus, human herpes viruses, vaccinia virus, Hepatitis B virus, and a combination thereof. In particular embodiments, the enveloped virus is a SARS-CoV-2 variant selected from B.1.351 (“South African Variant) or B.1.1.7 (“UK variant”).

In particular embodiments, the non-enveloped virus treated with the lipid agents herein include Iridoviridae, Adenoviridae, Polyomaviridae, Papillomaviridae (with dsDNA); Circoviridae, Parvoviridae (with ssDNA); Reoviridae, Birnavirus, and viruses belonging to the family Picornaviridae, Caliciviridae, Hepaviridae, Astroviridae, Nodaviridae (having positive sense strand ssRNA) and the like. For example, Enterorviruses of the Picornaviridae family (e.g., Coxsackievirus, Enterovirus, Poliovirus, Echovirus), Hepatoviruses (e.g., Hepatitis A virus), Rhinoviruses (e.g., Rhinovirus); Calipoviridae Sapoviruses (e.g., Sapovirus); Astroviridae mastrovirus genus (e.g., human astrovirus); Papillomaviridae papillomavirus genus (e.g., papillomavirus); Polyomaviridae polyomavirus genus (e.g., polyomavirus); Mastadenovirus (e.g., human adenovirus); Reoviridae, rotavirus (e.g., rotavirus); Caliciviridae, norovirus. In addition, the treated virus is from the genus Besivirus (for example, cat calicivirus) of the Caliciviridae family is known as a pathogenic virus of mammals other than humans, and the beta-nodavirus genus of the Nodaviridae family (viral neuronecrosis virus (NNV: Nervous virus) Necrosis Virus), etc.; Viruviridae, aquavirnavirus genus (Infectious pancreatic necrosis virus, etc.); Reoviridae, Aquareovirus genus; Iridoviridae, Ranavirus genus (Madiylid virus (RSBI: Red Sea) Bream Iridovirus)), or Parvoviridae (Infectious Hypodermal and Hematopoietic Necrosis Virus, etc.); and Dicistroviridae (mastroberry disease virus, Taura syndrome virus, etc.)

In certain embodiments, the virus treated with the lipid agents herein is a respiratory virus. Examples of respiratory viruses include, but are not limited to, influenza virus, respiratory syncytial virus (RSV), parainfluenza viruses, metapneumovirus, rhinovirus, coronaviruses, adenoviruses, and bocaviruses.

In certain embodiments, the lipid agent is a glycosphingolipid. Glycosphingolipids are a subtype of glycolipids containing the amino alcohol sphingosine. They may be considered as sphingolipids with an attached carbohydrate. Glycosphingolipids are a group of lipids (more specifically, sphingolipids) and are often part of the cell membrane. They are composed of a hydrophobic ceramide part and a glycosidically bound carbohydrate part. Glycosphingolipids have been found in lower and higher eukaryotic sources. They are composed of a glycan structure attached to a lipid tail that contains the sphingolipid ceramide. The basic structure for a glycosphingolipid is a monosaccharide, usually glucose or galactose, attached directly to a ceramide molecule and resulting in, respectively, glucosylceramide (glucocerebroside; GlcCer) or galactosylceramide (galactocerebroside; GalCer). The core glycan structure may be extended by additional monosaccharides. This combination structure results in an amphiphilic molecule with a hydrophilic carbohydrate region and a hydrophobic lipid region. In addition to variations in the structure of the glycan, the ceramide structure may also show variation. The fatty acid attached to the sphingosine may contain carbon chain lengths from C14 to C24 (e.g., C14, C15, C16, C17, C18, C19, C20, C21, C22, C23, or C24) and vary in degree of unsaturation and/or hydroxylation.

In certain embodiments, the lipid agent herein is a ganglioside. A ganglioside is a molecule composed of a glycosphingolipid (ceramide and oligosaccharide) with one or more sialic acids (e.g. n-acetylneuraminic acid, NANA) linked on the sugar chain. NeuNAc, an acetylated derivative of the carbohydrate sialic acid, makes the head groups of gangliosides anionic at pH 7, which distinguishes them from globosides. Structures of common gangliosides include:
GM2-1=aNeu5Ac(2-3)bDGalp(1)bDGalNAc(1)bDGalNAc(1)bDGlcp(1-1)Cer;
GM3=aNeu5Ac(2-3)bDGalp(1-4)bDGlcp(1-1)Cer;
GM2,GM2a=N-Acetyl-D-galactose-beta-1,4-[N-Acetylneuraminidate-alpha-2,3-]-Galactose-beta-1,4-glucose-alpha-ceramide;
GM2b=aNeu5Ac(2-8)aNeu5Ac(2-3)bDGalp(1-4)bDGlcp(1-1)Cer;
GM1,GM1a=bDGalp(1-3)bDGalNAc[aNeu5Ac(2-3)]bDGalp(1-4)bDGlcp(1-1)Cer;
asialo-GM1,GA1=bDGalp(1-3)bDGalpNAc(1-4)bDGalp(1-4)bDGlcp(1-1)Cer;
asialo-GM2,GA2=bDGalpNAc(1-4)bDGalp(1-4)bDGlcp(1-1)Cer;
GM1b=aNeu5Ac(2-3)bDGalp(1-3)bDGalNAc(1-4)bDGalp(1-4)bDGlcp(1-1)Cer;
GD3=aNeu5Ac(2-8)aNeu5Ac(2-3)bDGalp(1-4)bDGlcp(1-1)Cer;
GD2=bDGalpNAc(1-4)[aNeu5Ac(2-8)aNeu5Ac(2-3)]bDGalp(1-4)bDGlcp(1-1)Cer;
GD1a=aNeu5Ac(2-3)bDGalp(1-3)bDGalNAc(1-4)[aNeu5Ac(2-3)]bDGalp(1-4)bDGlcp(1-1)Cer;
GD1alpha=aNeu5Ac(2-3)bDGalp(1-3)bDGalNAc(1-4)[aNeu5Ac(2-6)]bDGalp(1-4)bDGlcp(1-1)Cer;
GD1b=bDGalp(1-3)bDGalNAc(1-4)[aNeu5Ac(2-8)aNeu5Ac(2-3)]bDGalp(1-4)bDGlcp(1-1)Cer;
GT1a=aNeu5Ac(2-8)aNeu5Ac(2-3)bDGalp(1-3)bDGalNAc(1-4)[aNeu5Ac(2-3)]bDGalp(1-4)bDGlcp(1-1)Cer;
GT1,GT1b=aNeu5Ac(2-3)bDGalp(1-3)bDGalNAc(1-4)[aNeu5Ac(2-8)aNeu5Ac(2-3)]bDGalp(1-4)bDGlcp(1-1)Cer;
OAc-GT1b=aNeu5Ac(2-3)bDGalp(1-3)bDGalNAc(1-4)aXNeu5Ac9Ac(2-8)aNeu5Ac(2-3)]bDGalp(1-4)bDGlcp(1-1)Cer;
GT1c=bDGalp(1-3)bDGalNAc(1-4)[aNeu5Ac(2-8)aNeu5Ac(2-8)aNeu5Ac(2-3)]bDGalp(1-4)bDGlcp(1-1)Cer;
GT3=aNeu5Ac(2-8)aNeu5Ac(2-8)aNeu5Ac(2-3)bDGal(1-4)bDGlc(1-1)Cer;
GQ1b=aNeu5Ac(2-8)aNeu5Ac(2-3)bDGalp(1-3)bDGalNAc(1-4)[aNeu5Ac(2-8)aNeu5Ac(2-3)]bDGalp(1-4)bDGlcp(1-1)Cer; and
GGal=aNeu5Ac(2-3)bDGalp(1-1)Cer;
   where:
aNeu5Ac=N-acetyl-alpha-neuraminic acid;
aNeu5Ac9Ac=N-acetyl-9-O-acetylneuraminic acid;
bDGalp=beta-D-galactopyranose;
bDGalpNAc=N-acetyl-beta-D-galactopyranose;
bDGlcp=beta-D-glucopyranose; and
Cer=ceramide (general N-acylated sphingoid).

In certain embodiments, the lipid agent herein is, or comprises a ceramide. Ceramides are a family of waxy lipid molecules. A ceramide is composed of sphingosine and a fatty acid. Ceramides are found in high concentrations within the cell membrane of eukaryotic cells, since they are component lipids that make up sphingomyelin, one of the major lipids in the lipid bilayer. Contrary to previous assumptions that ceramides and other sphingolipids found in cell membrane were purely supporting structural elements, ceramide can participate in a variety of cellular signaling: examples include regulating differentiation, proliferation, and programmed cell death (PCD) of cells.

In some embodiments, the lipid agent herein comprises a galactosylceramide. A galactosylceramide, or galactocerebroside is a type of cerebroside composed of a ceramide with a galactose residue at the 1-hydroxyl moiety.

In some embodiments, the lipid agent herein comprises a glucosylceramide. Glucosylceramides (glucocerebrosides) are any of the cerebrosides in which the monosaccharide head group is glucose.

In some embodiments, the lipid agent herein comprises a sphingomyelin. Sphingomyelin (SPH) is a type of sphingolipid found in animal cell membranes, especially in the membranous myelin sheath that surrounds some nerve cell axons. It usually is composed of phosphocholine and ceramide, or a phosphoethanolamine head group; therefore, sphingomyelins can also be classified as sphingophospholipids. In humans, SPH represents ˜85% of all sphingolipids, and typically make up 10-20 mol % of plasma membrane lipids. Sphingomyelin is composed of a phosphocholine head group, a sphingosine, and a fatty acid. It is one of the few membrane phospholipids not synthesized from glycerol. The sphingosine and fatty acid can collectively be categorized as a ceramide. This composition allows sphingomyelin to play significant roles in signaling pathways: the degradation and synthesis of sphingomyelin produce important second messengers for signal transduction.

In certain embodiments, the lipid agent is incorporated into, or on, an artificial carries, such as liposomes, nanoparticles (e.g., PLGA), dendrimers, quantum dots, polymersomes, gold nanoparticles, carbon nanotubes, or mixtures thereof. In other embodiments, the artificial carrier comprises a multilamellar vesicle (MLV), a small unilamellar liposome vesicle (SUV), micelle, and/or large unilamellar vesicles (LUV). Exemplary methods of generating liposomes incorporating sulfatides are found in, for example, Suzuki et al., FEBS Letters 553 (2003) 355-359); Perino et al., Biol. Cell (2011) 103, 319-331; and Watarai et al. J. Biochem. 108, 507-509 (1990); all of which are herein incorporated by reference in their entirety and specifically for the description of making sulfatide liposomes.

The lipid agents recited herein may be formulated in pharmaceutical formulations and/or medicaments. For example, for injection, the pharmaceutical formulation and/or medicament may be a powder suitable for reconstitution (e.g., at a hospital or pharmacy) with an appropriate solution (e.g., IV solution, such as Lactated Ringers solution). Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. In certain embodiments, the sulfatides are mixed with an organic polar solvent (e.g., DMSO). In certain embodiments, the sulfatides are mixed with a buffer (e.g., phosphate buffered saline).

The lipid agents of the invention may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of the lipid agents herein by inhalation.

Ordinarily, an aqueous aerosol is made by formulating an aqueous solution or suspension of the lipid agents together with conventional pharmaceutically acceptable carriers and stabilizers. The carriers and stabilizers vary with the requirements of the particular compound, but typically include nonionic surfactants (e.g., TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver the lipid agents of the invention.

Aerosols containing lipid agents for use according to the present invention are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the lipid compound and a suitable powder base such as lactose or starch. Delivery of aerosols of the present invention using sonic nebulizers is advantageous because nebulizers minimize exposure of the agent to shear, which can result in degradation of the compound.

For nasal administration, the pharmaceutical formulations and medicaments with the lipid agents may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the lipid agent maybe formulated in oily solutions or as a gel. For administration of nasal aerosol, any suitable propellant may be used including compressed air, nitrogen, carbon dioxide, or a hydrocarbon based low boiling solvent.

In certain embodiments, the pharmaceutical formulations are administered orally, in the form of a pill capsule, gel-cap, or the like. In some embodiments, the oral administration is 10-1500 mg of sulfatides per kilogram of subject (e.g., 10 . . . 75 . . . 100 . . . 125 . . . 150 . . . 200 . . . 250 . . . 300 . . . 400 . . . 500 . . . 650 . . . 800 . . . 1000 . . . 1500 mg/kg). In certain embodiments, provided herein are pills or capsules containing sulfatides (e.g., only type of sulfatide is present) or other lipid agents (e.g., only one type of lipid agent is present). In particular embodiments, such pills or capsules are stored at about −22 to −15 degrees Celsius (e.g., in a home freezer). In certain embodiments, such pills or capsules are in a jar (e.g., and a user stores the jar in their home freezer). In particular embodiments, the pills or capsules (e.g., softgels) have an enteric coating.

Dosage forms for the topical (including buccal and sublingual) or transdermal or oral administration of lipid agents of the invention include powders, sprays, pills, gel-caps, ointments, pastes, creams, lotions, gels, solutions, and patches. The lipid agents herein may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pill or capsule herein comprises a gelatin encapsulated dosage form (e.g., a softgel). In certain embodiments, the gelatin encapsulation of the lipid agent herein is composed of gelatin, glycerin, water, and optionally caramel. In particular embodiments, the pills and capsules herein are coated with an enteric coating (e.g., to avoid the acid environment of the stomach, and release most of the lipid agent in the small intestines of a subject). In some embodiments, the enteric coating comprises a polymer barrier that prevents its dissolution or disintegration in the gastric environment, thus allowing the lipid agents herein (e.g., sulfatides) to reach the small intestines. Examples of enteric coatings include, but are not limited to, Methyl acrylate-methacrylic acid copolymers; Cellulose acetate phthalate (CAP); Cellulose acetate succinate; Hydroxypropyl methyl cellulose phthalate; Hydroxypropyl methyl cellulose acetate succinate (hypromellose acetate succinate); Polyvinyl acetate phthalate (PVAP); Methyl methacrylate-methacrylic acid copolymers; Shellac; Cellulose acetate trimellitate; Sodium alginate; Zein; COLORCON, and an enteric coating aqueous solution (ethylcellulose, medium chain triglycerides [coconut], oleic acid, sodium alginate, stearic acid) (e.g., coated softgels). Additional enteric coatings are described in Hussan et al., IOSR Journal of Pharmacy, e-ISSN: 2250-3013, p-ISSN: 2319-4219, Volume 2 Issue 6, November-December 2012, PP. 05-11, herein incorporated by references in its entirety, and particularly for its description of enteric coatings.

Transdermal patches may be employed herein, and have the added advantage of providing controlled delivery of a compound of the invention to the body. Such dosage forms can be made by dissolving or dispersing the lipid agent in the proper medium. Absorption enhancers can also be used to increase the flux of the lipid agent across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

Specific dosages of the lipid agents herein may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

In certain embodiments, the lipids agents (e.g., only type, or multiple types) herein are mixed with carrier lipids for form liposomes. In some embodiments, the carrier lipids in the liposomes are one or more of the following: DDAB, dimethyldioctadecyl ammonium bromide; DPTAP (1,2-dipalmitoyl 3-trimethylammonium propane); DHA; prostaglandin, N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammonium chloride; DOGS, dioctadecylamidoglycylspermine; DC-cholesterol, 3.beta.-[N—(N′,N′-dimethylaminoethane)carbamoyl]cholesterol; DOSPA, 2,3-dioleoyloxy-N-(2(sperminecarboxamido)-ethyl)-N,N-dimethyl-1-propanami-nium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); beta-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl) diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butan-ediammonium iodide; 1-[2-acyloxy)ethyl]2-alkyl (alkenyl)-3-(2-hydroxyethyl-) imidazolinium chloride derivatives such as 1-[2-(9(Z)-octadecenoyloxy)eth-yl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyeth-yl)imidazolium chloride (DMTIM) (e.g., as described in Solodin et al. (1995) Biochem. 43:13537-13544, herein incorporated by reference); 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE) (e.g., as described in Felgner et al. (1994) J. Biol. Chem. 269:2550-2561, herein incorporated by reference in its entirety). Many of the above-mentioned lipids are available commercially from, e.g., Avanti Polar Lipids, Inc.; Sigma Chemical Co.; Molecular Probes, Inc.; Northern Lipids, Inc.; Roche Molecular Biochemicals; and Promega Corp. In certain embodiments, the non-sulfatide lipids are one or more of the following: distearoyl phosphatidyl choline (DSPC), dimyristoyl phosphatidylcholine (DMPC), dipalmitoyl phosphatidylcholine (DPPC), palmitoyl oleoyl phosphatidylcholine (POPC), palmitoyl stearoyl phosphatidylcholine (PSPC), egg phosphatidylcholine (EPC), hydrogenated or non-hydrogenated soya phosphatidylcholine (HSPC), or sunflower phosphatidylcholine. In some embodiments, the non-sulfatide lipids are one or more of the following: distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), egg phosphatidylethanolamine (EPE), and transphosphatidylated phosphatidylethanolamine (t-EPE), which can be generated from various natural or semisynthetic phosphatidylcholines using phospholipase D.

In certain embodiments, the lipid agents herein (e.g., a sulfatide, a sulfatide analog, a ceramide, a lipid moiety comprising a ceramide, a sulfoglycolipid, a sulfogalactolipid, a glycosphingolipid, a seminolipid, a glucosylceramide, a sphingomyelin, or a galactosylceramide) and optionally a secondary therapeutic (e.g., other antiviral or drug to treat effects of viral infection) are administered in a cycle of less than about 3 weeks, about once every two weeks, about once every 10 days or about once every week. One cycle can comprise the administration of an lipid agent herein and optionally a second active agent (e.g., another antiviral) by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In particular embodiments, courses of treatment can be administered concurrently to a subject, i.e., individual doses of the lipid agents herein and secondary therapeutic are administered separately yet within a time interval such that the lipid agent herein can work together with the additional therapeutic agent. For example, one component can be administered once per week in combination with the other components that can be administered once every two weeks or once every three weeks. In other words, the dosing regimens are carried out concurrently even if the therapeutics are not administered simultaneously or during the same day.

### EXAMPLE

### Example 1

### Sulfatide Toxicity Study

This example describes methods employed to test the toxicity of sulfatides in vivo. Two control mice (#1 and #2) were injected daily with a carrier solution of 0.5% Tween 80 in PBS (phosphate buffered saline). Two treated mice (#3 and #4) were injected daily with 200 microliters of 1.5 mg/ml sulfatides (bovine sulfatide mixture, Cayman, no. 24323) in the same carrier solution, for a total daily dose of 300 ug of sulfatides (which is 15 mg/kg). Daily injection were conducted for six days. Blood was drawn from the mice on the last day and sent away for an IDEXX analysis. The IDEXX results are shown in Table 1 below.

TABLE 1

| Clinical Chemistry | | | | | |
|---|---|---|---|---|---|
| | Normal | Control Mice | | Treated Mice | |
| | Range | 1 | 2 | 3 | 4 |

| | | | | |
|---|---|---|---|---|
| ALP (U/L) | 88 | 88 | 73 | 115 |
| AST (U/L) | 58 | 44 | 46 | 63 |
| ALT (U/L) | 28 | 28 | 24 | 44 |
| Amylase (U/L) | 552 | 587 | 557 | 657 |
| Albumin (g/dL) | 2.8 | 2.8 | 3.0 | 3.2 |
| Total Billirubin (mg/dL) | 0.2 | 0.1 | 0.2 | 0.2 |
| Total Protein (g/dL) | 4.6 | 4.5 | 4.7 | 5.3 |
| Globulin (g/dL) | 1.8 | 1.7 | 1.7 | 2.1 |
| Billirubin - Conjugated | 0.0 | 0.0 | 0.0 | 0.0 |
| (mg/dL) | | | | |
| BUN (mg/dL) | 16 | 23 | 17 | 22 |
| Creatinine (mg/dL) | 0.1¹ | 0.1¹ | 0.1¹ | 0.1¹ |
| Cholesterol (mg/dL) | 108 | 67 | 61 | 78 |
| Glucose (mg/dL) | 224 | 124 | 149 | 146 |
| Calcium (mg/dL) | 108 | 67 | 61 | 78 |
| Phosphorus (mg/dL) | 5.7 | 6.0 | 6.2 | 6.6 |
| Chloride (mmol/L) | 111 | 111 | 110 | 109 |
| Potassium (mmol/L) | 4.6 | 4.3 | 4.3 | 5.1 |
| ALB/GLOB ratio | 1.6 | 1.6 | 1.8 | 1.5 |
| Sodium (mmol/L) | 149 | 153 | 151 | 151 |
| BUN/Creatinine Ratio | 160.0 | 230.0 | 170.0 | 220.0 |
| Billirubin - Unconjugated | 0.2 | 0.1 | 0.2 | 0.2 |
| (mg/dL) | | | | |
| NA/K Ratio | 32 | 36 | 35 | 30 |
| Hemolysis Index | + | Normal | Normal | Normal |
| Lipemia Index | Normal | Normal | Normal | Normal |

The results in Table 1 shows that this concentration of sulfatides was well tolerated by the mice. The control and treated mice were also found to be very active on day 6.

### Example 2

### Cell Culture Viral Inhibition Testing of Sulfatides Against 12 Viruses

This example describes testing the antiviral effect of sulfatides (sulfatide mixture; Cayman Chemical Co., item 24323) against twelve viruses using cytopathic effect (CPE)-based inhibition assay and two vesicular stomatitis virus (VSV)-based pseudovirus expressing filovirus glycoproteins (GP) using Luciferase-based inhibition assay as outlined in the table below. Cytotoxic effect was assessed in parallel.

TABLE 2

| | | Stock Titer | | Cell | Assay |
|---|---|---|---|---|---|
| Virus | Strain | (PFU/mL) | MOI | line | Readout |
| Adenovirus | 5 | 2.22E+06 | 0.030 | Vero | CPE |
| (ADV) | | | | cells | (ELISA) |
| Chikungunya | 181/25 | 7.27E+07 | 0.025 | Vero | CPE |
| virus (CHKV) | | | | cells | (Crystal |
| | | | | | Violet) |
| Human | AD169 | 1.26E+04 | 0.013 | MRC-5 | CPE |
| Cytomegalo- | | | | | (ELISA) |
| virus (hCMV) | | | | | |
| Dengue virus | D2Y98P | 1.80E+07 | 0.20 | Vero | CPE |
| serotype 2 | | | | Cells | (ELISA) |
| (DENV-2) | | | | | |
| Herpes simplex | MacIntyre | 7.24E+04 | 0.025 | Vero | CPE |
| virus subtype 1 | | | | Cells | (Crystal |
| (HSV-1) | | | | | Violet) |
| Herpes simplex | MS | 5.30E+05 | 0.015 | Vero | CPE |
| virus subtype 2 | | | | Cells | (Crystal |
| (HSV-2) | | | | | Violet) |
| Influenza virus | B/Lee/1940 | 2.15E+05 | 0.017 | MDCK | CPE |
| (INFV) B | | | | cells | (ELISA) |
| Respiratory | A2 | 3.54E+06 | 0.015 | HEp-2 | CPE |
| Syncytial Virus | | | | cells | (ELISA) |
| (RSV) | | | | | |
| Venezuelan | 181/29 | 2.89E+08 | 0.030 | Vero | CPE |
| Equine | | | | cells | (Crystal |
| Encephalitis | | | | | Violet) |
| Virus (VEEV) | | | | | |
| VSV- | Mayinga | N/A | N/A | Vero | Luciferase |
| Ebolavirus- | | | | cells | (RLU) |
| GB (EBOV) | | | | | |
| VSV- | Angola | N/A | N/A | Vero | Luciferase |
| Marburgvirus- | | | | cells | (RLU) |
| GP (MARV) | | | | | |
| Zika Virus | RSS13025 | 4.68E+06 | 0.030 | Vero | CPE |
| (ZIKV) | | | | Cells | (Crystal |
| | | | | | Violet) |

N/A = Not Applicable; pseudoviruses stocks are titrated and approximately 20,000 RLU are used per well.

### Assays Employed

### CPE Inhibition Assay

Vero, MDCK and HEp-2 cells were seeded on Day −1 in 96-well tissue culture plates at 1.00, 1.80, and 1.30E+04 cells per well, respectively. Eight two-fold serial dilutions of the sulfatides were prepared in infection media and added in triplicate to the cells and incubated for one-hour at room temperature. Each virus was prepared at its specific multiplicity of infection (MOI; see Table 2) and added to the sulfatides/cells mix. Virus only and cells only wells were also added. All infected cells were incubated at 37° C. and 5% CO₂ except for MDCK cells, which were infected with INFV incubated at 35° C. After the appropriate time of incubation, cells were immuno-stained (hCMV, DENV, INFV, RSV and ADV) or stained with crystal violet (CHKV, HSV-1, HSV-2 and ZIKV). Optical density was read for calculation of 50 percent inhibition concentration (IC50) of the TA using XLfit dose response model.

### Luciferase-Based Inhibition Assay (for Pseudovirus)

Vero cells were seeded in black 96-well plates on Day −1 at 5.00E+04 cells per well Eight 2-fold serial dilutions of the sulfatides were prepared in infection medium, added in triplicate to Vero cells and incubated for one-hour at room temperature. Approximately 20,000 RLU of VSV-EBOV and VSV-MARV were prepared and added to the TA/cells mix. Virus only and cells only were added. Plates were incubated for 24-hours at 37° C. and 5% CO₂. Firefly Luciferase activity was detected using the Bright-Glo™ Assay System kit (Promega). Fifty (50) percent inhibition concentration (IC50) was calculated using XLfit dose response model.

### Cytotoxicity Assay

Vero, MDCK and HEp-2 cells were seeded on Day −1 in 96-well tissue culture plates in parallel of the inhibition assay as described above. Sulfatide dilutions were prepared and incubated with cells for one-hour incubation to mimic inhibition assay conditions as described above. Additional infection media was then added to match inhibition assay volumes. Cells only and medium only wells were also added. For each virus, cytotoxicity and inhibition assays were terminated on the same day. Cells were lysed for evaluation of the ATP content using Promega's Cell Titer Glo kit. The luciferase luminescence in relative light units (RLU) was read and 50 percent cytotoxicity concentration (CC50) was calculated using XLfit dose response model.

### Sulfatide Formulation

Table 3 shows the sulfatide formulation employed.

TABLE 3

| | | | Working | | |
|---|---|---|---|---|---|
| | | Stock | Stock | Start | |
| | | Concentra- | Concentra- | Concentra- | Dilution |
| TA | Diluent | tion (mM) | tion (μM) | tion (μM) | factor |
| Sulfatide | DMSO | ¹250 | ²25,000 | 250 | 2-fold |
| Mixture | | | | | |

¹50 mg of TA (sulfatide mixture) was dissolved into 241 μL of 100% DMSO for a stock concentration of 207.525 μg/mL or 250 mM.
²1:10 working stock concentration at 25 mM was prepared in DMSO.

### Results

Table 4A below shows a summary of results for each virus.

Table 4B shows the IC50 values for the viruses in mg/ml and ug/ml.

TABLE 4B

| | | mg/ml | μg/mL |
|---|---|---|---|
| | | concentration | concentration |
| Virus | IC₅₀ (μM) | IC50 | IC50 |
| ADV | 9.639 | 0.008 | 8 |
| CHKV | 41.09 | 0.034 | 34 |
| DENV-2 | ND | | ND |
| HSV-1 | 32.79 | 0.027 | 27 |
| HSV-2 | 10 | 0.008 | 8 |
| INFV B | 859.5 | 0.713 | 713 |
| RSV | 0.8639 | 0.001 | 1 |
| VEEV | ND | | ND |
| EBOV | 48.54 | 0.040 | 40 |
| MARV | 21.34 | 0.018 | 18 |
| ZIKV | 35.88 | 0.030 | 30 |
| hCMV | 11.41 | 0.009 | 9 |

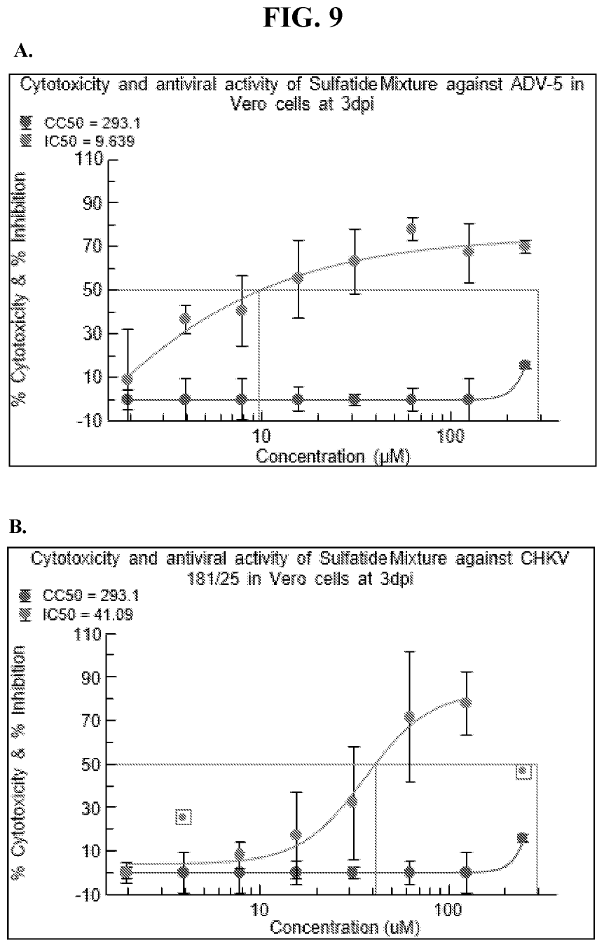

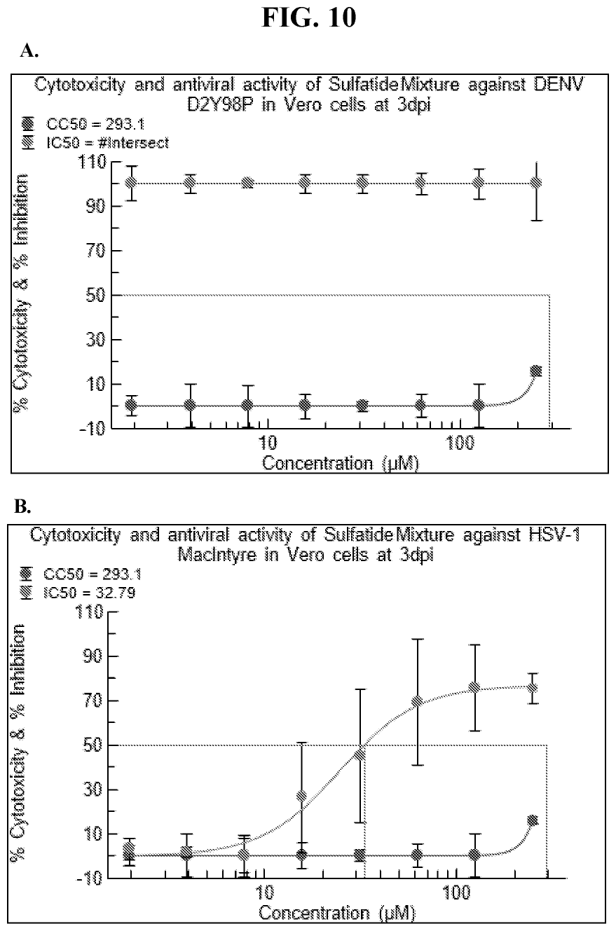

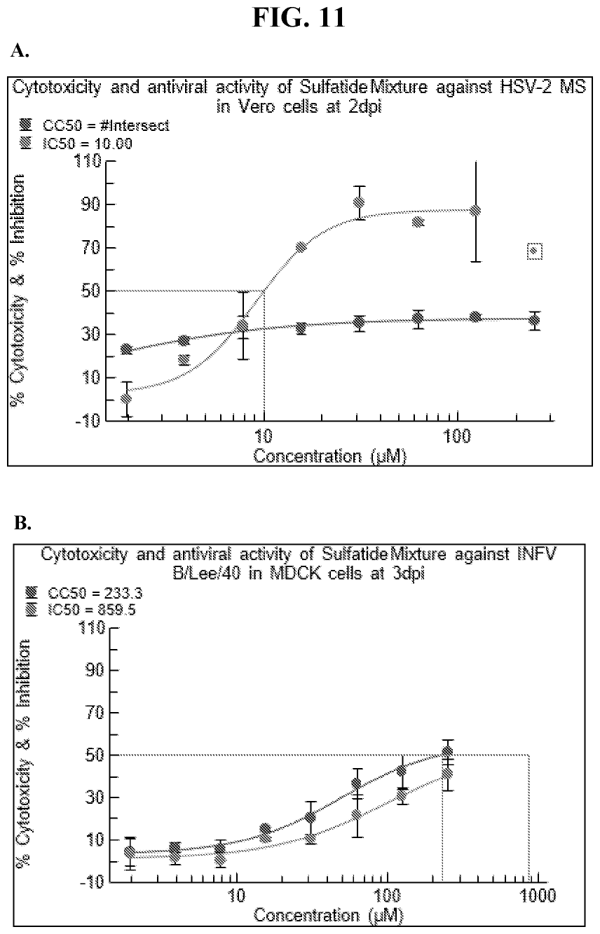

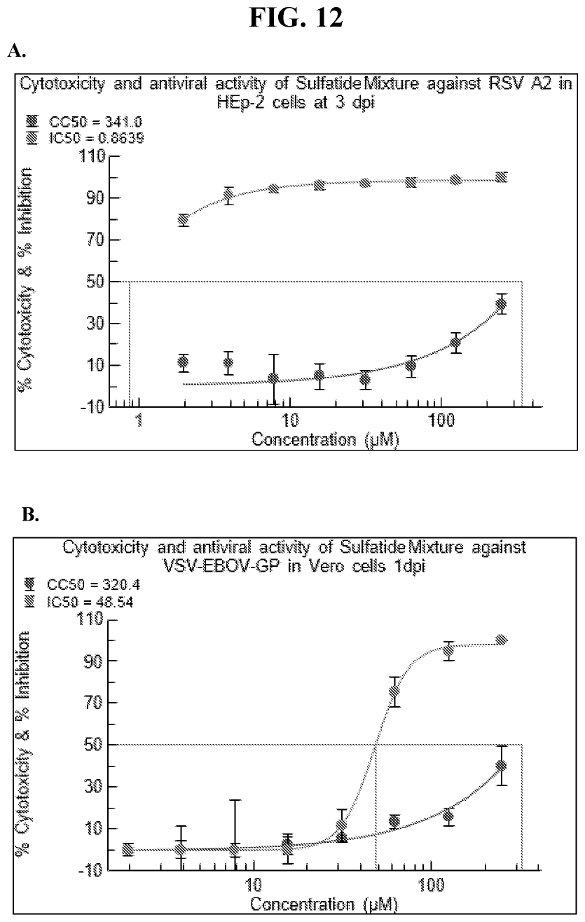

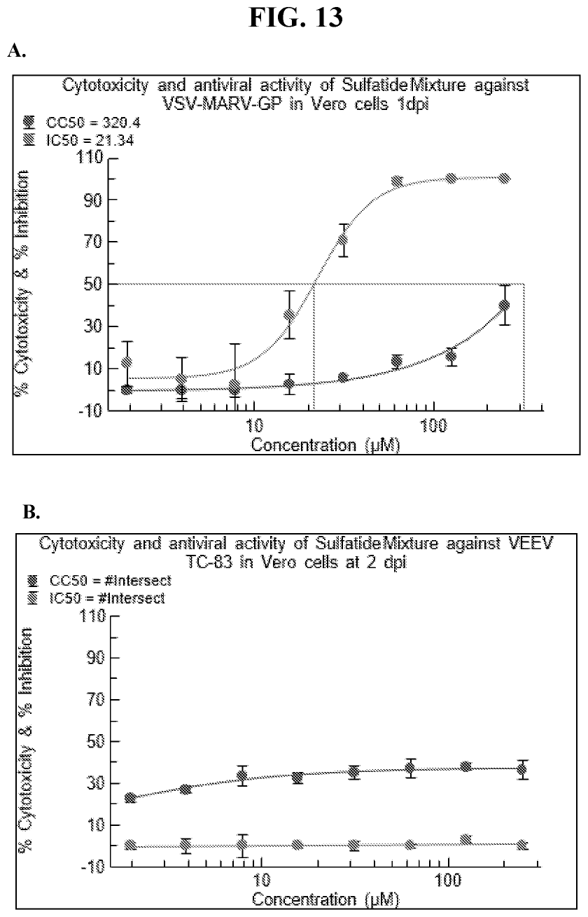

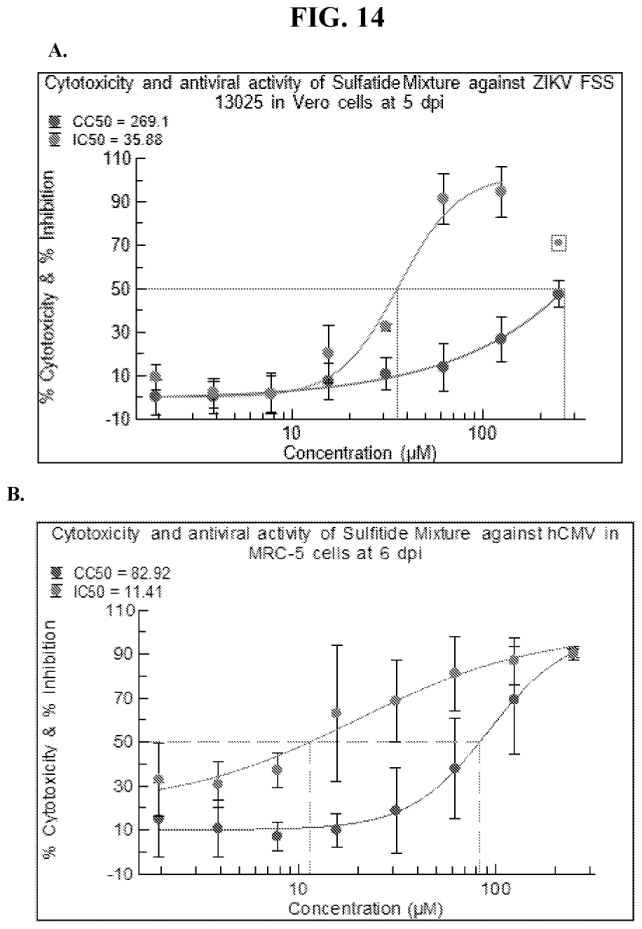

The cytotoxicity and antiviral activity of the sulfatide mixture against each of the twelve viruses are shown in FIGS. 9-14. FIG. 9A shows cytotoxicity results (blue line) and antiviral inhibition results (green line) for ADV-5 (Adenovirus), and FIG. 9B shows cytotoxicity results (blue line) and antiviral inhibition results (green line) for CHKV (Chikungunya virus). FIG. 10A shows cytotoxicity results (blue line) and antiviral inhibition results (green line) for DENV (Dengue virus), and FIG. 10B shows cytotoxicity results (blue line) and antiviral inhibition results (green line) for HSV-1 (Herpes simplex virus, type 1). FIG. 11A shows cytotoxicity results (blue line) and antiviral inhibition results (green line) for HSV-2 (Herpes simplex virus, type 2), and FIG. 11B shows cytotoxicity results (blue line) and antiviral inhibition results (green line) for INFV B (Influenza A). FIG. 12A shows cytotoxicity results (blue line) and antiviral inhibition results (green line) for RSV (Respiratory syncytial virus), and FIG. 12B shows cytotoxicity results (blue line) and antiviral inhibition results (green line) for pseudo EBOV (Pseudovirus VSV-EBOLA virus). FIG. 13A shows cytotoxicity results (blue line) and antiviral inhibition results (green line) for pseudo MARV (VSV-Marburg virus), and FIG. 13B shows cytotoxicity results (blue line) and antiviral inhibition results (green line) for VEEV (Venezuelan equine encephalitis virus). FIG. 14A shows cytotoxicity results (blue line) and antiviral inhibition results (green line) for pseudo ZIKV (Zika virus). FIG. 14B shows cytotoxicity results (blue line) and antiviral inhibition results (green line) for hCMV (human cytomegalovirus). Eleven out of the twelve viruses were inhibited by sulfatides (all except for VEEV). Of these eleven inhibited viruses, all of them had low IC50 values except influenza B.

### Example 3

### Cell Culture Viral Inhibition Testing of Sulfatides Against SARS-CoV-2

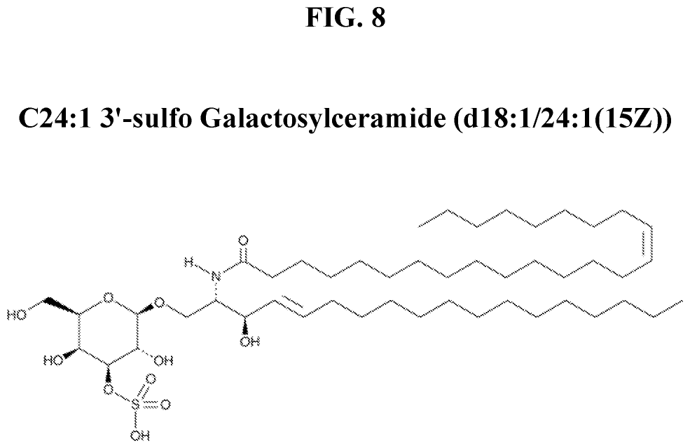

This example describes testing the antiviral effect of sulfatides (sulfatide C24:1; Cayman Chemical Co., item 24865; shown in FIG. 8) against SARV-CoV-2. Antiviral assays against live SARS-CoV-2 were performed with the MEX-BC2/2020 strain, which contains the D614G mutation in the spike protein (GISAID database ID: EPI_ISL_747242). The sulfatide was provided as a solid, from which a 20 mg/mL DMSO stock was prepared and kept at −20° C. until used. The sulfatide was assessed in parallel for antiviral and viability assays. For this Example, Vero E6 cells were utilized to evaluate the antiviral activity of the sulfatide against SARS-CoV-2. Sulfatide was pre-incubated first with target cells for 1 h at 37° C. before infection with SARS-CoV-2. Following pre-incubation, cells were challenged with viral inoculum. Putative inhibitor sulfatide was present in the cell culture for the duration of the infection (96 hours), at which time a Neutral Red uptake assay was performed to determine the extent of the virus-induced cytopathic effect (CPE). Prevention of the virus-induced CPE was used as a surrogate marker to determine the antiviral activity of the test-item against SARS-CoV-2. Controls wells were also included with GS-441524, an inhibitor of SARS-CoV-2 and the main plasma metabolite of the polymerase inhibitor remdesivir (GS-5734). A cell viability assay with Vero E6 uninfected cells was set up in parallel in a separate plate and for the same duration of the infectivity assay (96 h). Cell viability was also determined with the Neutral Red method. Eight dilutions of the sulfatide sample was tested in duplicates for the antiviral and viability assays. The sulfatide was submitted to two-fold serial dilutions starting at 100 μg/mL. When possible, IC50 (antiviral) and CC50 (inhibition of viability) values of the test-item were determined using GraphPad Prism software.

### Results Summary

### Antiviral Activity of Sulfatide 24:1

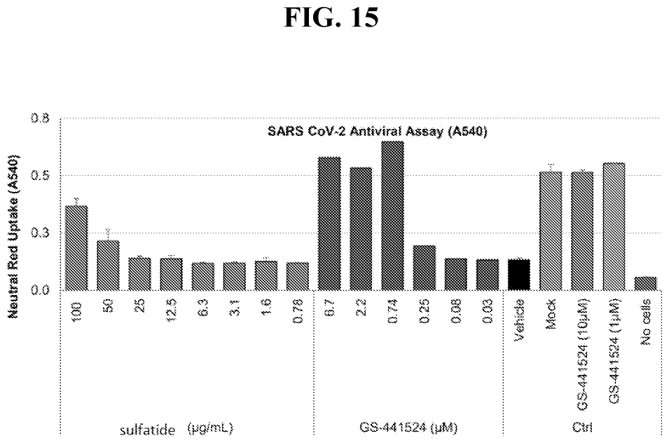

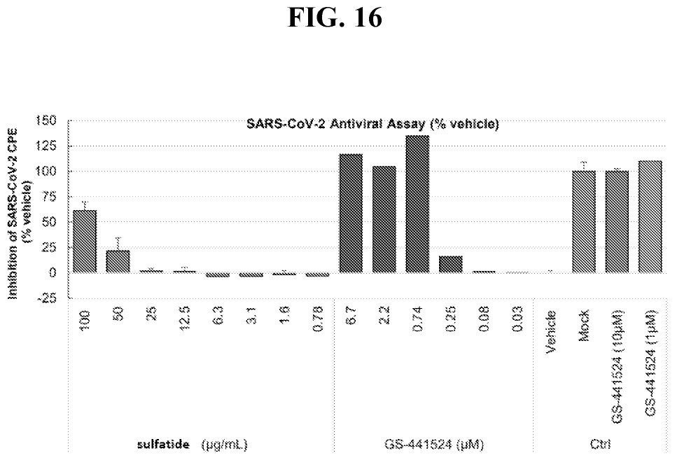

The sulfatide prevented the virus-induced cytopathic effect (CPE) in a dose-dependent manner at the two highest concentrations tested, 50 μg/mL and 100 μg/mL. The IC50 value obtained for the sulfatide was 84.9 μg/mL (FIG. 16). The sulfatide also displayed a dose-dependent trend of cytotoxicity in the viability assay with uninfected cells, suggesting that the compound-induced cytotoxicity may have partially interfered with the prevention of the virus-induced CPE. IC50, CC50 and selectivity indices (S.I.) for the test-item are summarized in Table 5 further below. The control SARS-CoV-2 inhibitor GS-441524 completely prevented the virus-induced CPE at concentrations at or above 0.74 μM (FIGS. 15 and 16). The antiviral effect of GS-441524 was also confirmed by microscopic evaluation of the cell monolayers. The IC50 value of GS-441524 against SARS-CoV-2 was approximately 0.28 μM.

### Control Inhibitors and Quality Controls

Quality controls for the infectivity assays were performed on every plate to determine: i) signal to background (S/B) values; ii) inhibition by known inhibitors of SARS-CoV-2 (for antiviral assay), and iii) variation of the assay, as measured by the coefficient of variation (C.V.) of all data points. All controls worked as anticipated for each assay, including the control GS-441524, a known inhibitor of SARS-CoV-2 infection that prevented completely the virus-induced CPE of the infected cells. Overall variation of duplicates in the antiviral assay was 8.8%. Overall variation in the viability assays was 5.7%. The ratio of signal-to-background (S/B) for the antiviral assay was 3.9-fold, determined by comparing the A540 nm values in uninfected (“mock”) cells with that observed in cells challenged with SARS-CoV-2 in the presence of vehicle alone. When comparing the signal in uninfected cells to the signal in “no-cells” background wells, the S/B ratio of the antiviral assay was 9.1-fold. For the viability assay the S/B (“no cells” value) was 5.5-fold

Table 5 shows the IC50 (antiviral), and CC50 (cytotoxicity) values for the sulfatide and GS-441254. Signal-to-background ratios (S/B), average coefficients of variation (C.V.), and selectivity indices (S.I.) are shown. The average C.V. was determined for all replicate test-item data-points in the CPE assays (antiviral) and the viability assays (cytotoxicity in uninfected cells). When cell viability did not reach 50% at the highest concentration tested, CC50 values is shown as greater than the highest concentration tested.

TABLE 5

| | Live SARS-CoV-2 Antiviral Assay | | | Cytotoxicity Assay | | | |
|---|---|---|---|---|---|---|---|
| | IC50 | | | CC50 | | | |
| Sample | (μg/mL) | S/B¹ | C.V.² | (μg/mL) | S/B¹ | C.V.² | S.I.³ |
| Sulfatide | 84.9 | 3.9 | 8.8 | >100 | 5.5 | 5.7 | >1.2 |
| 24:1 | | | | | | | |
| GS- | ~0.28⁴ | 3.9 | 8.8 | n.t. | n.t. | n.t. | n.d. |
| 441524 | | | | | | | |
| (μM) | | | | | | | |

¹Signal to background in antiviral assays was calculated by dividing the signal in uninfected cells (“mock-infected”), by the signal in infected cells. For viability assays the signal in vehicle (medium only) was divided by the signal in the “no cells.”
²C.V. for the antiviral and cytotoxicity assays were calculated as the average of C.V. values determined for all replicate sulfatide data points.
³The selectivity index (S.I.) is calculated by dividing the CC50 value by the IC50 value.
⁴Value was estimated by the GraphPad Prism Software.
R2 = 0.977;
n.d.: not determined;
n.t.: not tested

1 Signal to background in antiviral assays was calculated by dividing the signal in uninfected cells (“mock-infected”), by the signal in infected cells. For viability assays the signal in vehicle (medium only) was divided by the signal in the “no cells.” 2 C.V. for the antiviral and cytotoxicity assays were calculated as the average of C.V. values determined for all replicate sulfatide data points. 3 The selectivity index (S.I.) is calculated by dividing the CC50 value by the IC50 value. 4 Value was estimated by the GraphPad Prism Software. R2=0.977: n.d.: not determined: n.t.: not tested

### Experimental Procedure—SARS-CoV-2 Antiviral Assay

To evaluate antiviral activity against SARS-CoV-2, the isolate MEX-BC2/2020 carrying a D614G mutation in the viral spike protein (GISAID database ID: EPI_ISL_747242) was used. A CPE-based antiviral assay was performed by infecting Vero E6 cells in the presence or absence of sulfatide and control. Infection of cells leads to significant cytopathic effect and cell death after 4 days of infection. In this assay, reduction of CPE in the presence of inhibitors was used as a surrogate marker to determine the antiviral activity of the tested items. Viability assays to determine test-item-induced loss of cell viability was monitored in parallel using the same readout (Neutral Red), but utilizing uninfected cells incubated with the test-items.

Vero E6 cells were maintained in DMEM with 10% fetal bovine serum (FBS), hereby called DMEM10. Twenty four hours after cell seeding, test samples were submitted to serial dilutions with DMEM2 in a different plate. Then, media was removed from cells, and serial dilutions of test-items were added to the cells and incubated for 1 h at 37° C. in a humidified incubator. After cells were pre-incubated with test-items, then cultures were challenged with SARS-CoV-2 resuspended in DMEM with 2% FBS (DMEM2). The amount of viral inoculum was previously titrated to result in a linear response inhibited by antivirals with known activity against SARS-CoV-2. Cell culture media with the virus inoculum was not removed after virus adsorption, and test-items and virus were maintained in the media for the duration of the assay (96 h). After this period, the extent of cell viability was monitored with the neutral red (NR) uptake assay.

The virus-induced CPE was monitored under the microscope after 3 days of infection. After 4 days cells were stained with neutral red to monitor cell viability. Viable cells incorporate neutral red in their lysosomes. The uptake of neutral red relies on the ability of live cells to maintain the pH inside the lysosomes lower than in the cytoplasm, a process that requires ATP. Inside the lysosome the dye becomes charged and is retained. After a 3 h incubation with neutral red (0.017%), the extra dye is washed away, and the neutral red is extracted from lysosomes by incubating cells for 15 minutes with a solution containing 50% ethanol and 1% acetic acid. The amount of neutral red is estimated by measuring absorbance at 540 nm in a plate reader.

Test-item was evaluated in duplicates using serial 2-fold dilutions starting at 100 μg/mL. Controls included uninfected cells (“mock-infected”), and infected cells to which only vehicle was added. Some cells were treated with GS-441524 (1 μM and 10 μM or a full-dose response curve in single data points). GS-441524 is the main metabolite of remdesivir, a broad-spectrum antiviral that blocks the RNA polymerase of SARS-CoV-2.

### Data Analysis of CPE-Based Antiviral Assay

The average absorbance at 540 nm (A540) observed in infected cells (in the presence of vehicle alone) was calculated, and then subtracted from all samples to determine the inhibition of the virus induced CPE. Data points were then normalized to the average A540 signal observed in uninfected cells (“mock”) after subtraction of the absorbance signal observed in infected cells. In the neutral red CPE-based assay, uninfected cells remained viable and uptake the dye at higher levels than non-viable cells. In the absence of antiviral agent the virus-induced CPE kills infected cells and leads to lower A540 (this value equals 0% inhibition). By contrast, incubation with the antiviral agent (GS-441524) prevents the virus induced CPE and leads to absorbance levels similar to those observed in uninfected cells. Full recovery of cell viability in infected cells represent 100% inhibition of virus replication

Experimental Procedure—Cytotoxicity Assays with Uninfected Cells

### Viability Assay (Neutral Red Uptake Method) to Assess Test-Item-Induced Cytotoxicity

Uninfected cells were incubated with eight concentrations of sulfatide or control inhibitors dilutions using starting the same doses indicated for the antiviral assay. The incubation temperature and duration of the incubation period mirrored the conditions of the prevention of virus-induced CPE assay, and cell viability was evaluated with the neutral red uptake method but this time utilizing uninfected cells. The extent of viability was monitored by measuring absorbance at 540 nm. When analyzing the data, background levels obtained from wells with no cells were subtracted from all data-points. Absorbance readout values were given as a percentage of the average signal observed in uninfected cells treated with vehicle alone.

### QC and Analysis of Cytotoxicity Data

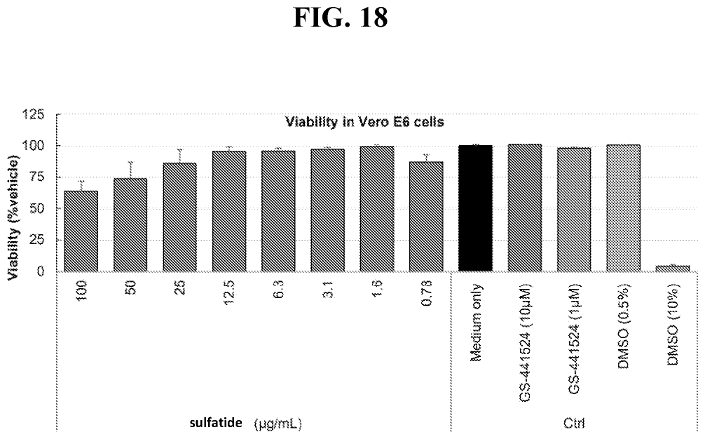

The average signal obtained in wells with no cells was subtracted from all samples. Readout values were given as a percentage of the average signal observed in uninfected cells treated with vehicle alone (DMEM2). The signal-to-background (S/B) obtained was 5.5-fold. DMSO was used as a cytotoxic compound control in the viability assays. DMSO blocked cell viability by more than 95% when tested at 10% (FIG. 18).

### Results—SARS-CoV-2 Antiviral Assays

Table 6 shows protection from SARS-CoV-2-induced CPE by the sulfatide (A540). Raw values represent A540 levels obtained determining the uptake of neutral red into viable cells. Infected cells develop CPE after four days of infection and displayed significantly reduced absorbance levels. Duplicates A540 values are shown for each test-item concentration. All samples were infected except those indicated as “mock.” Varying concentrations of GS-441524 were also evaluated in each plate. Test-item concentrations are in μg/mL and GS-441524 are shown in μM.

TABLE 6
Raw OD- Absorbance (540 nm)

| | | | | | | | | | | | | | |
|---|---|---|---|---|---|---|---|---|---|---|---|---|---|
| Conc. | 100 | 50 | 25 | 12.5 | 6.25 | 3.1 | 1.6 | 0.78 | Vehicle | Mock | GS- | GS- | |
| (μg/mL) | | | | | | | | | | | 441524 | 441524 | No |
| | | | | | | | | | | | (10 μM) | (1 μM) | cells |
| sulfatide | 0.390 | 0.180 | 0.134 | 0.127 | 0.115 | 0.122 | 0.137 | 0.121 | 0.130 | 0.496 | 0.507 | 0.554 | 0.058 |
| | 0.344 | 0.250 | 0.146 | 0.18 | 0.121 | 0.116 | 0.115 | 0.118 | 0.136 | 0.519 | 0.522 | 0.553 | 0.055 |
| Conc. | 6.7 | 2.2 | 0.74 | 0.25 | 0.08 | 0.03 | | | 0.146 | 0.578 | | | |
| (μM) | | | | | | | | | | | | | |
| GS- | 0.578 | 0.533 | 0.648 | 0.194 | 0.137 | 0.133 | | | 0.124 | 0.521 | | | |
| 441524 | | | | | | | | | | | | | |
| | | | | | | | | | 0.137 | 0.493 | | | |
| | | | | | | | | | 0.123 | 0.481 | | | |

FIG. 15 shows the results of inhibition by the sulfatide of SARS-CoV-2-induced CPE (A540). Cell viability was monitored to determine the virus induced-CPE. Data is shown as raw A540 values in wells containing Vero E6 cells infected in the presence of either vehicle alone or varying concentrations of test-item (average of duplicates with standard deviation). Uninfected cells are shown as “Mock.” Background levels are shown in wells without cells (“no cells”). Also included, the dose-response observed with GS-441524 (single data-points).

Table 7 shows SARS-CoV-2 CPE Assay (percentage values), showing the inhibition of the SARS-CoV-2 (MEX-BC2/2020) induced CPE in Vero E6 cells. Prevention of the virus induced CPE was used as a surrogate marker to determine the extent of replication of SARS-CoV-2. The lower levels of neutral red uptake in infected cells in the presence of vehicle alone are indicative of no inhibition of the virus-induced CPE. Complete inhibition (100%) results in A540 levels equal to those observed in mock-infected cells (with vehicle alone). To obtain percentage inhibition values, the average A540 in cells infected in the absence of test-item (“vehicle”) was subtracted from all values, and then these values were normalized to those obtained for uninfected cells (“mock”). Uninfected cells in the presence of vehicle alone are equal to 100% inhibition. Percentage inhibition is shown for each test condition. All samples shown below were infected except those indicated as “mock.” Some samples are treated with GS-441524, known antiviral agent with activity against SARS-CoV-2. Sulfatide concentrations and controls are shown in microgram per milliliter. Data shown for test-item represents the average and standard deviation of duplicates. For uninfected cells (“mock”) and “vehicle” the standard deviation was derived from six replicates.

TABLE 7
Inhibition of SARS-CoV-2 (MEX-BC2/2020) Virus-Induced CPE (% mock)

| | | | | | | | | | | | | |
|---|---|---|---|---|---|---|---|---|---|---|---|---|
| Conc. | 100 | 50 | 25 | 12.5 | 6.25 | 3.1 | 1.6 | 0.78 | Vehicle | GS- | GS- | Mock |
| (μg/mL) | | | | | | | | | | 441524 | 441524 | |
| | | | | | | | | | | (10 μM) | (1 μM) | |
| sulfatide | 61.3 ± 8.5 | 21.6 ± 13.0 | 1.9 ± 2.2 | 1.3 ± 3.9 | −3.8 ± 1.1 | −3.6 ± 1.1 | −1.7 ± 4.1 | −3.4 ± 0.6 | 0.0 ± 2.3 | 100.0 ± | 110.2 ± | 100.0 ± |
| Conc. | 6.7 | 2.2 | 0.74 | 0.25 | 0.08 | 0.03 | | | | 2.8 | 0.2 | 9.1 |
| (μM) | | | | | | | | | | | | |
| GS- | 116.6 | 104.8 | 134.9 | 16.1 | 1.1 | 0.09 | | | | | | |
| 441524 | | | | | | | | | | | | |

FIG. 16 shows inhibition by the test item (sulfatide) of the CPE mediated by SARS-CoV-2 (percentage values). Values show the inhibition of the SARS-CoV-2 induced CPE, as a surrogate marker for virus replication. Data was analyzed as shown in Table 7, with values normalized to the A540 values observed in uninfected cells after subtraction of the average absorbance observed in infected cells in the presence of vehicle. Values in uninfected cells (“mock”) are included for comparison (100% inhibition). Data plotted for test-item shows the average and standard deviation of duplicates. Also included, the dose-response observed with GS-441524 (single data-points).

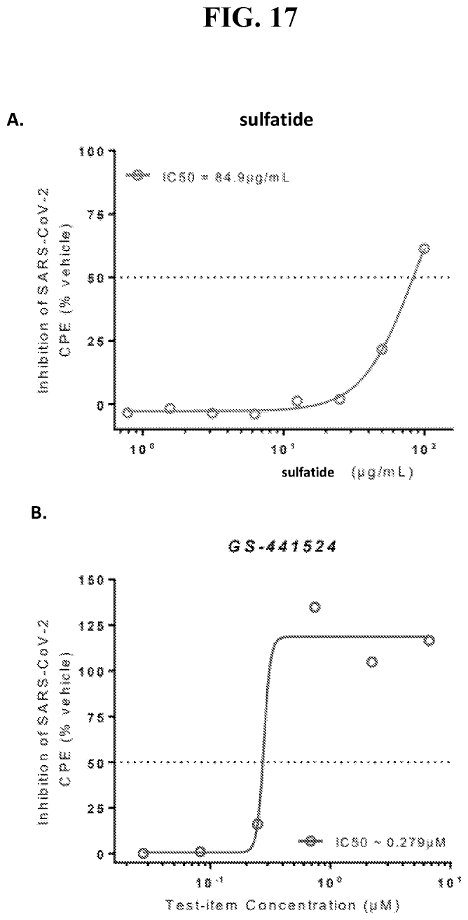

FIG. 17 shows IC50 values for inhibition of SARS-CoV-2 CPE by sulfatide (FIG. 17A) and GS-441524 (FIG. 17B). Values indicate the percentage inhibition of the CPE induced by live SARS-CoV-2 (MEX-BC2/2020), as compared to samples incubated with no test-item (vehicle alone). Test-item results show the average of duplicate data points. Bottom graphs show single data points for GS-441524. Data was modeled to a sigmoidal function using GraphPad Prism software fitting a dose-response curve with a variable slope (four parameters). IC50 value for GS-441524 curve was estimated by the GraphPad Prism Software with an R2=0.977. IC50 values are also summarized in Table 5.

Table 8, A and B shows the viability of Vero E6 cells in the presence of sulfatide as determined by the neutral red uptake assay. Vero E6 cells (uninfected) were incubated for 4 days in the presence of different concentrations of test-item, or with vehicle alone (medium only). For each data point the individual raw data is shown (A540). Table 8B shows raw data values for the vehicle alone, control inhibitor GS-441524 (1 μM and 10 μM) and the cytotoxic agent (DMSO at 10% and 0.5%).

TABLE 8A
Viability of Uninfected Vero E6 Cells (A540)

| | | | | | | | | |
|---|---|---|---|---|---|---|---|---|
| Conc. | 100 | 50 | 25 | 12.5 | 6.25 | 3.1 | 1.6 | 0.78 |
| (μg/mL) | | | | | | | | |
| sulfatide | 0.370 | 0.425 | 0.468 | 0.486 | 0.483 | 0.487 | 0.495 | 0.457 |
| | 0.325 | 0.350 | 0.405 | 0.466 | 0.470 | 0.479 | 0.487 | 0.424 |

TABLE 8B

| | |
|---|---|
| Controls | Viability (A450) |

| | | |
|---|---|---|
| No Cells (background) | 0.090 | 0.089 |
| Medium only (vehicle) | 0.498 | 0.492 |
| | 0.495 | 0.496 |
| | 0.496 | 0.486 |
| GS-441524 (10 μM) | 0.498 | 0.498 |
| GS-441524 (1 μM) | 0.488 | 0.484 |
| DMSO (0.5%) | 0.497 | 0.495 |
| DMSO (10%) | 0.102 | 0.110 |

Table 9A and 9B, show viability of Vero E6 cells determined by the neutral red uptake assay (percentage values). Values indicate the percent viability remaining in uninfected Vero E6 after a 4-day treatment with sulfatide. Values are shown as percentage of the viability observed in samples incubated with vehicle alone (Medium only). Data represents the mean and standard deviation of duplicates for test-item. Vehicle values were derived from six replicates. Table 9B shows the percentage viability observed in cells treated with tissue culture medium in the absence of sulfatide (Medium only), control inhibitor GS-441524 (1 μM and 10 μM), or with the cytotoxic agent (DMSO 0.5% and 10%).

TABLE 9A
Viability of Uninfected Vero E6 Cells (% vehicle)

| | | | | | | | | |
|---|---|---|---|---|---|---|---|---|
| Conc. | 100 | 50 | 25 | 12.5 | 6.25 | 3.1 | 1.6 | 0.78 |
| (μg/mL) | | | | | | | | |
| sulfatide | 63.8 ± 7.9 | 73.7 ± 13.1 | 85.8 ± 11.0 | 95.6 ± 3.5 | 95.7 ± 2.3 | 97.3 ± 1.4 | 99.3 ± 1.4 | 89.8 ± 5.8 |

TABLE 9B

| Controls | Viability (A450) |
|---|---|
| No Cells (background) | 0.0 ± 0.2 |
| Medium only (vehicle) | 100.0 ± 1.1 |
| GS-441524 (10 μM) | 101.0 ± 0.0 |
| GS-441524 (1 μM) | 98.1 ± 0.7 |
| DMSO (0.5%) | 100.5 ± 0.3 |
| DMSO (10%) | 4.1 ± 1.4 |

FIG. 18 shows viability in uninfected Vero E6 cells (percentage values). Results show the extent of cell viability as determined by the neutral red uptake assay (A540) after 4 days. Data is normalized to the values observed in cells in the absence of sulfatide (“vehicle,” medium only). Test-item results show the average of duplicate data points with the standard deviation (s.d.). Average and standard deviation values for cells treated with vehicle (medium only) are derived from six replicates.

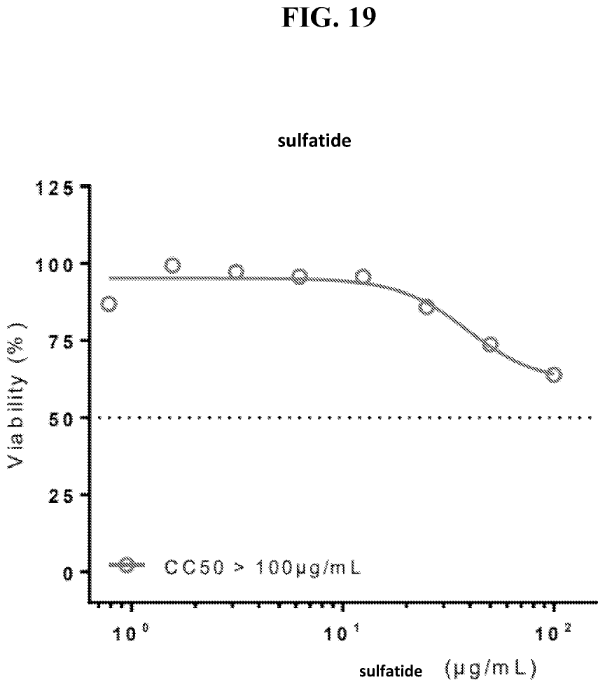

FIG. 19 shows CC50 values for Vero E6 cell viability in the presence of sulfatide (percentage values). Values indicate the percent viability estimated as percentage of that observed in samples incubated with vehicle alone (medium). Results show the average of duplicate data points. Bottom graph shows overlapping curves from sulfatide. Data was adjusted to a sigmoid function when possible, and CC50 values were calculated using GraphPad Prism software fitting a dose-response curve with a variable slope (four parameters). CC50 values are also summarized in Table 5.

### REFERENCES

[1] U.S. Pat. No. 5,486,536 to Ward et al. entitled “Sulfatides as Anti-Inflammatory Compound,” filed Aug. 15, 1994.
[2] Mulligan et al., Anti-inflammatory effects of sulfatides in selectin-dependent acute lung injury, Int Immunol. 1995 July; 7(7): 1107-13.
[3] Squadrito et al., Effect of Sulfatide on Acute Lung Injury During Endotoxemia in Rats, Life Sciences, Vol. 65, No. 24, pp. 2541-2552, 1999.
[4] Zhang et al., Sulfatide-activated type II NKT cells prevent allergic airway inflammation by inhibiting type I NKT cell function in a mouse model of asthma, Am J Physiol Lung Cell Mol Physiol 301: L975-L984, 2011.
[5] Cao, COVID-19: immunopathology and its implications for therapy, Nature Reviews Immunology (2020), published April 9th.
[6] Sundell et al., Sulfatide Administration Leads to Inhibition of HIV-1 Replication and Enhanced Hematopoiesis, Journal of Stem Cells, 5(1):33-42, 2010.
[7] Campbell et al., Lipid rafts and HIV-1: from viral entry to assembly of progeny virions, Journal of Clinical Virology 22 (2001) 217-227.
[8] Suzuki et al., (1996) Sulphatide binds to human and animal influenza A viruses, and inhibits the viral infection. Biochem. J. 318, 389-393.
[9] Verma et al., Host Lipid Rafts Play a Major Role in Binding and Endocytosis of Influenza A Virus, Viruses 2018, 10, 650, 1-11.
[10] Perino et al., Role of sulfatide in vaccinia virus infection, Biol. Cell (2011) 103, 319-331.
[11] Fantini et al., (article in press), International Journal of Antimicrobial Agents, https://doi.org/10.1016/j.ijantimicag.2020.105960.
[12] Chung et al., Vaccinia virus penetration requires cholesterol and results in specific viral envelope proteins associated with lipid rafts, J Virol. 2005 February; 79(3): 1623-34.
[13] Isaac et al., Sulfatide with short fatty acid dominates in astrocytes and neurons, FEBS Journal 273 (2006) 1782-179.
[14] Svennerholm et al., Changes in the fatty acid composition of cerebrosides and sulfatides of human nervous tissue with age, J. of Lipid Res., 1968, Vol. 9, 215-225.
[15] Mirzaian et al., Quantification of sulfatides and lysosulfatides in tissues and body fluids by liquid chromatography-tandem mass spectrometry, Journal of Lipid Research Volume 56, 2015.
[16] Guo et al., Effects of hypertension and antihypertensive treatments on sulfatide levels in serum and its metabolism, Hypertension Research volume 42, pages 598-609 (2019).
[17] Buschard et al., Low serum concentration of sulfatide and presence of sulfated lactosylceramid are associated with Type 2 diabetes. The Skaraborg Project, Diabet Med. 2005 September; 22(9):1190-8.
[18] Yuzhe et al., Serum sulfatide abnormality is associated with increased oxidative stress in hemodialysis patients, Hemodialysis Int., Volume 19, Issue 3 (2015).
[19] Hu et al., Serum sulfatides as a novel biomarker for cardiovascular disease in patients with end-stage renal failure, Glycoconj J. 2007 December; 24(9):565-71.
[20] Suzuki et al., Inhibition of influenza A virus sialidase activity by sulfatide, FEBS Letters 553 (2003) 355-359.
[21] Watarai et al., Inhibitory Effect of Liposomes Containing Sulfatide or Cholesterol Sulfate on Syncytium Formation Induced by Bovine Immunodeficiency Virus-Infected Cells, J. Biochem. 108, 507-509 (1990).
[22] Vigant et al., PLOS Pathogens, 1 Apr. 2013, Volume 9, Issue 4.
[23] Verma et al., J. of Drug Targeting, 2020, No. 10, 1046-1052.
[24] U.S. Pat. No. 8,044,029
[25] Takahashi et al., J. Biochem. 2012; 152(4):373-380.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1-81. (canceled)

82. A method of treating a subject infected with an enveloped or non-enveloped virus comprising:
administering a composition to a subject, or providing said composition to said subject such that said subject administers said composition to themselves;
wherein said subject is infected with an enveloped or non-enveloped virus;
wherein said composition comprises:
i) a plurality of at least one type of naked sulfatide; and/or
ii) a plurality of at least one type of sulfatide and non-sulfatide carrier lipids which are combined in the form of a plurality of sulfatide-containing liposomes; and
wherein said composition contains only one or two types of sulfatide, and is detectably free of other types of sulfatides.

83. The method of claim 82, wherein said virus is HIV.

84. The method of claim 82, wherein said non-sulfatide carrier lipids comprise phospholipids.

85. The method of claim 82, wherein said sulfatide-containing liposomes further comprise cholesterol.

86. The method of claim 82, wherein said at least one sulfatide is selected from the group consisting of: 18:0(2R—OH) Sulfo GalCer; 18:0(2S—OH) Sulfo GalCer; C24:1 Mono-Sulfo Galactosyl(B) Ceramide (d18:1/24:1); C17 Mono-Sulfo Galactosyl(β) Ceramide (d18:1/17:0); C12 Mono-Sulfo Galactosyl(B) Ceramide (d18:1/12:0); C12 Di-Sulfo Galactosyl(β) Ceramide (d18:1/12:0); C24 Mono-Sulfo Galactosyl(B) Ceramide (d18:1/24:0); C24:1 Mono-sulfo galactosyl (alpha) ceramide (d18:1/24:1); C19:0-Sulfatide; N-Nonadecanoyl-sphingosyl-beta-D-galactoside-3-sulfate; and mixtures of two thereof.

87. The method of claim 82, wherein said subject is a human.

88. The method of claim 82, wherein said administering is such that said subject receives about 0.01-50 mg, of said lipid agent per kilogram of said subject.

89. The method of claim 82, wherein said administering is intravenous administration or via said subject's airway, or the subject administers said composition to themselves orally or via an inhaler.

90. The method of claim 82, wherein said subject administers said composition to themselves orally, and wherein said composition comprises a pill or capsule, and/or wherein said subject receives about 5-1500 mg of said lipid agent per kilogram of said subject per day.

91. The method of claim 82, wherein said at least one type of sulfatide is the only type of sulfatide present in said composition.

92. The method of claim 82, wherein the fatty acid chain length of said sulfatide is selected from the group consisting of: 16, 17, 18, 19, 20, 21, 22, 23, or 24.

93. A system, kit, or article of manufacture comprising:
a) a composition comprising:
i) a plurality of at least one type of naked sulfatide; and/or
ii) a plurality of at least one type of sulfatide and non-sulfatide carrier lipids which are combined in the form of a plurality of sulfatide-containing liposomes; and
wherein said composition contains only one or two types of sulfatide, and is detectably free of other types of sulfatides; and
and
b) a container selected from the group consisting of:
i) an IV fluid solution bag,
ii) a syringe vial,
iii) a syringe,
iv) a sterile shipping container configured for shipping powder or liquid,
v) an airway administration device, and
vi) an orally ingestible dosage form.

94. The system, kit, or article of manufacture of claim 93, wherein the fatty acid chain length of said sulfatide is selected from the group consisting of: 16, 17, 18, 19, 20, 21, 22, 23, and 24.

95. A composition comprising:
a) a plurality of at least one type of naked sulfatide; and/or
b) a plurality of at least one type of sulfatide and non-sulfatide carrier lipids which are combined in the form of a plurality of sulfatide-containing liposomes; and
wherein said composition contains only one or two types of sulfatide, and is detectably free of other types of sulfatides.

96. The composition of claim 95, wherein the fatty acid chain length of said sulfatide is selected from the group consisting of: 16, 17, 18, 19, 20, 21, 22, 23, and 24.
